(19) United States
(12) Patent Application Publication
(10) Pub. No.: US 2023/0183419 A1
(43) Pub. Date: Jun. 15, 2023

(54) Biodegradable Copolymers and Nanofibrous Scaffold Thereof

(71) Applicant: THE REGENTS OF THE UNIVERSITY OF MICHIGAN, Ann Arbor, MI (US)

(72) Inventors: Renato S. Navarro, Ann Arbor, MI (US); Peter X. Ma, Ann Arbor, MI (US)

(21) Appl. No.: 17/919,834

(22) PCT Filed: Apr. 22, 2021
(86) PCT No.: PCT/US21/28545

§ 371 Date: Oct. 19, 2022

Related U.S. Application Data
(60) Provisional application No. 63/014,484, filed on Apr. 23, 2020.

(51) Int. Cl. C08G 63/08; A61L 27/18; A61L 27/58; A61L 27/50; A61L 27/56

(52) U.S. Cl. CPC C08G 63/08; A61L 27/18; A61L 27/58; A61L 27/507; A61L 27/56; A61L 2430/34

(57) ABSTRACT

Provided herein are biodegradable copolymers, methods of lactone polymerization, nanofibrous scaffolds, and methods of regenerating tissue.

### CROSS-REFERENCE TO RELATED APPLICATIONS

This application claims the benefit under 35 U.S.C. § 119(e) of U.S. Provisional Patent Application No. 63/014,484, filed on Apr. 23, 2020, the entirety of which is hereby incorporated by reference.

### STATEMENT OF US GOVERNMENT SUPPORT

This invention was made with government support under grant number HL114038 and HL136231, awarded by the National Institutes of Health. The government has certain rights in the invention.

### BACKGROUND

Biodegradable polymers are widely used in biomedical applications, such as tissue engineering scaffolds, drug releasing micro-/nano-particles, and implants. However, for many of such applications, a reasonably high molecular weight (HMW) is often necessary for such polymers to be processed into the desired physical forms and/or to possess the desired mechanical and functional properties. Unfortunately, synthesizing HMW polymers (such as above 100 kDa) often requires high temperature, extended reaction time periods, and specialized reaction vessels that are costly.

### SUMMARY

One aspect of the present disclosure provides a biodegradable copolymer comprising a structure of:

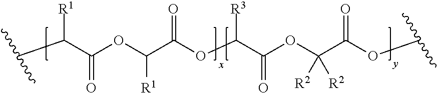

wherein each R¹ is independently selected from C₁-C₂₂ alkyl, C₂-C₂₂ alkenyl, C₅-C₁₂ cycloalkyl, C₅-C₁₂cycloalkenyl, Ar¹, carboxyl, ester, amino, thiol, halo, C₁-C₂₂ haloalkyl, SH, OH, or a first functional group of a click chemistry reactive pair; the geminal R² groups as a pair, together with the carbon atom to which they are attached, form a five- to twelve-member cyclic or bicyclic group, or each R² is independently selected from H, C₁-C₂₂ alkenyl, C₅-C₈ cycloalkenyl, carboxyl, amido, C₁-C₂₂ haloalkenyl, OH, SH, and Ar¹, or a first functional group of a click chemistry reactive pair; R³ is selected from C₁-C₂₂ alkyl, C₂-C₂₂alkenyl, C₅-C₁₂cycloalkyl, C₅-C₁₂cycloalkenyl, Ar¹, carboxyl, ester, amino, thiol, halo, C₁-C₂₂ haloalkyl, OH, or a first functional group of a click chemistry reactive pair; each Ar¹ is independently selected from C₆-C₂₂ aryl or a 5-12 membered heteroaryl comprising from 1 to 3 ring heteroatoms selected from O, N, and S; and each of x and y is an integer; with a proviso that when the geminal R² groups as a pair, together form

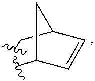 [SMILES: *CC1C=CC(*)C1]

each occurrence of R¹ are C₁-alkyl, and R³ is C₁-alkyl, then the weight average molecular weight of the biodegradable copolymer is about 35 kDa or more.

Another aspect of the present disclosure provides a method of lactone polymerization comprising: admixing: (a) a cooled mixture comprising lactone monomers and a solvent, the mixture having a temperature of about −30° C. to about −110° C.; and (b) about 0.01 mol % to about 5 mol % of a guanidine derivative, based on the total mols of monomers, optionally in a solvent, to form the lactone polymer, wherein upon admixing, the concentration of lactone monomers is about 15 w/v % or less, based on the total volume of solvent.

Another aspect of the present disclosure provides a nanofibrous scaffold comprising biodegradable copolymer of the disclosure, polyspirolactide, or a combination thereof.

Another aspect of the present disclosure provides a method of regenerating tissue comprising implanting the nanofibrous scaffold of the disclosure in a tissue.

### BRIEF DESCRIPTION OF THE DRAWINGS

FIG. 1A-1E depicts the characterization of polyspirolactide (PSLA) and the precursor monomer, spirolactide: FIG. 1A shows a 500 MHz ¹H NMR spectrum of exomethylene lactide (a precursor to spirolactide) (1) in CDCl₃; FIG. 1B shows a 500 MHz ¹H NMR spectrum of spirolactide (2); FIG. 1C shows a 500 MHz ¹H NMR spectrum of polyspirolactide; FIG. 1D shows FTIR characterization depicting stretches of key functional groups of L-lactide, (1), (2), and PSLA; and FIG. 1E shows UV-Vis absorbance of L-lactide, (1), (2), and PSLA. FIG. 2A-2C depicts a fabrication of a PSLA/PLLA tubular nanofibrous scaffold of the disclosure that can be post-modified; FIG. 2A shows a fabrication method using a sugar template annealed on a mandrel for the fabrication of a tubular porous and interconnected network; FIG. 2B shows an overview and SEM characterization of the PSLA/PLLA layer that is highly porous, interconnected, and nanofibrous; and FIG. 2C shows a visualization of poly(caprolactone) (PCL) dense layer that is electrospun to provide enhanced mechanical support.

FIG. 3A-3G depicts post-modification and characterization of a PSLA/PLLA nanofibrous scaffold of the disclosure; FIG. 3A shows post-modification scheme of the PSLA of the copolymer with heparin via thiol-ene click chemistry; FIG. 3B shows FTIR spectra demonstrating pre and post-modification changes; FIG. 3C shows SEM visualization of tubular scaffold post modification; FIG. 3D shows confocal image of PLLA; FIG. 3E shows a confocal image of PSLA modified with FITC-PEG-SH; FIG. 3F shows the change of hydrophilicity by the change in water drop shape, contact angle quantification, and comparison of PLLA, unmodified PSLA, and modified PSLA films with increasing concentration of heparin; and FIG. 3G shows a graph depicting the contact angle of each.

FIG. 4A-4C depicts the degradation and mass loss of PLLA/PSLA scaffolds of the disclosure after heparin conjugation; FIG. 4A shows the PLLA/PSLA scaffold conjugated with heparin began to disintegrate at week 3 with all other groups intact at end of experiment while PLLA scaffolds show minimal degradation; FIG. 4B shows the quantification of scaffold mass loss over time due to heparin conjugation on PLLA/PSLA scaffolds compared to PLLA and PLLA/PSLA scaffolds with no heparin conjugation; and FIG. 4C shows SEM characterization of the scaffold at day 0 and the scaffold degradation at day 35.

FIG. 5A-5H depicts various images of post-operation of implanted scaffolds and a graph of the post-operation time versus inner diameter. FIGS. 5A and 5B show operative images for implanted scaffolds of the disclosure (FIG. 5A: immediately after implant; FIG. 5B: 3 months post-operation); FIGS. 5C-5E show the morphologies of nanofibrous vascular scaffolds of the disclosure before rat abdominal aortic interpositional implant (FIG. 5C), 3 months post-operation (FIG. 5D) and native rat abdominal aorta (FIG. 5E); FIGS. 5F and 5G show ultrasound images of the implanted scaffold 3 months post-operation; and Figure H is a graph of the comparison of native aorta vs tissue engineered blood vessels (TEBVs) over the 3-month period.

FIG. 6A depicts the comparison of vascular smooth muscle reconstruction at anastomosis and middle sites of implanted scaffolds of the disclosure by H&E staining; (A, B) 10× and 40× magnification from anastomosis site of scaffold at 1 week post-operation; (C, D) 10× and 40× magnification from middle site of scaffold at 1 week post-operation; (E, F) 10× and 40× magnification from anastomosis site of scaffold at 2 weeks post-operation; (G, H) 10× and 40× magnification from middle site of scaffold at 2 weeks post-operation; (I, J) 10× and 40× magnification from anastomosis site of scaffold at 1 month post-operation; (K, L) 10× and 40× magnification from middle site of scaffold at 1 month post-operation; (M, N) 10× and 40× magnification from anastomosis site of scaffold at 3 months post-operation; (0, P) 10× and 40× magnification from middle site of scaffold at 3 months post-operation; Figure B shows (Q, R) 10× and 40× magnification from rat's native aorta. (Scale bar: For A, C, E, G, I, K, M, O, Q=400 um; For B, D, F, H, J, L, N, P, R=40 um).

FIG. 7 depicts the comparison of vascular extracellular matrix reconstruction at sites of implanted scaffolds of the disclosure after implantation for 1 and 3 months; (A, B) 10× and 40× magnification from rat's native aorta by Masson trichrome staining; (C, D) 10× and 40× magnification from rat's native aorta by Verhoeff Van Gieson staining; (E, F) 10× and 40× magnification from implanted scaffold at 1 month post-op by Masson trichrome staining; (G, H) 10× and 40× magnification from implanted scaffold at 1 month post-op by Verhoeff Van Gieson staining; (I, J) 10× and 40× magnification from implanted scaffold at 3 months post-op by Masson trichrome staining; (K, L) 10× and 40× magnification from implanted scaffold at 3 months post-op by Verhoeff Van Gieson staining. (Scale bar: for A, C, E, G, J, K=400 um; For B, D, F, H, J, L=40 um).

FIG. 8A-8C depicts the immunofluorescence staining of smooth muscle cell marker SM22 indicating smooth muscle cell infiltration and rat aorta reconstruction in scaffolds of the disclosure, 1 mo post-op and 3 mo post-op; FIG. 8A is fluorescence staining wherein, A) shows fluorescence staining of SM22 in rat's native aorta; B) shows fluorescesce staining of SM22 in implanted scaffold at 1 mo post-op; and C) shows flourecense of SM22 in implanted scaffold at 3 mo post-op. FIG. 8B shows DAPI wherein, D) DAPI shows nuclei in rat native aorta; E) DAPI shows nuclei in implanted scaffold at 1 mo post-op; and F) DAPI shows nuclei in implanted scaffold at 3 mo post-op. FIG. 8C shows merged images of fluorescence and DAPI, wherein G) is a merged image of fluorescence and DAPI in rat native aorta; H) is a merged image of flourencesce and DAPI in implanted scaffold at 1 mo post-op; and I) is a merged of fluorescence and DAPI in implanted scaffold at 3 mo post-op. (Scale bar=40 um).

FIG. 9 depicts the immunohistochemical staining of endothelial marker vWF of implanted scaffolds of the disclosure; A) Rat native aorta; B) 2 weeks post-op; C) 1 month post-op; D) 3 months post-op. (Scale bar=40 um).

### DETAILED DESCRIPTION

Provided herein are biodegradable copolymers, methods of making biodegradable copolymers, and nanofibrous scaffolds made thereof. The biodegradable copolymers disclosed herein can comprise a structure of:

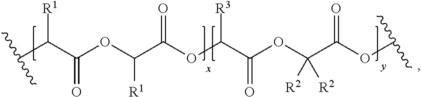

wherein each R¹ is independently selected from C₁-C₂₂ alkyl, C₂-C₂₂ alkenyl, C₅-C₁₂cycloalkyl, C₅-C₁₂ cycloalkenyl, Ar¹, carboxyl, ester, amino, thiol, halo, C₁-C₂₂ haloalkyl, SH, OH, or a first functional group of a click chemistry reactive pair; the geminal R² groups as a pair, together with the carbon atom to which they are attached, form a five- to twelve-member cyclic or bicyclic group, or each R² is independently selected from H, C₁-C₂₂ alkenyl, C₅-C₈ cycloalkenyl, carboxyl, amido, C₁-C₂₂ haloalkenyl, OH, SH, and Ar¹, or a first functional group of a click chemistry reactive pair; R³ is selected from C₁-C₂₂ alkyl, C₂-C₂₂ alkenyl, C₅-C₁₂ cycloalkyl, C₅-C₁₂cycloalkenyl, Ar¹, carboxyl, ester, amino, thiol, halo, C₁-C₂₂ haloalkyl, SH, OH, or a first functional group of a click chemistry reactive pair; each Ar¹ is independently selected from C₆-C₂₂ aryl or a 5-12 membered heteroaryl comprising from 1 to 3 ring heteroatoms selected from O, N, and S; and each of x and y is an integer; with a proviso that when the geminal R² groups as a pair, together form

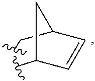 [SMILES: *CC1C=CC(*)C1]

each occurrence of R¹ are C₁-alkyl, and R³ is C₁-alkyl, then the weight average molecular weight of the biodegradable copolymer is about 35 kDa or more. Although the x and y segments are depicted in a block copolymer configuration, the depiction is not intended to be limiting; rather, the x and y segments can be provided in a block copolymer configuration, alternating copolymer configuration, or random copolymer configuration. In embodiments, the biodegradable copolymer has a random configuration of the x and y segments. In embodiments, the biodegradable copolymer has an alternating configuration of the x and y segments. In embodiments, the biodegradable copolymer has a block configuration of x and y segments.

The biodegradable copolymers of the disclosure can provide one or more advantages including, but not limited to: 1) having high molecular weights suitable for providing scaffolds with the necessary mechanical integrity to be used in a living body, for example, vascular grafting; and/or 2) having sufficient biodegradation, such as in vitro biodegradation, wherein, for example, the biodegradable copolymers disclosed herein can degrade in 10 mL of 1 M PBS, pH 7.4 and incubated at 37° C. to result in a 20-25% mass loss or more.

Also provided herein are methods of lactone polymerization. The methods of lactone polymerization, such as for the preparation of the biodegradable copolymer of the disclosure, can comprise two steps. The first step (step (a)) can comprise cooling a mixture of monomers in a solvent to a temperature of about −30° C. to about −110° C. to form a cooled mixture. The second step (step (b)) can comprise admixing the cooled mixture with a guanidine derivative to form a biodegradable polymer, wherein in the second step, the concentration of monomers is about 15 w/v % or less, based on the total volume of solvent, such as about 0.1 w/v % to about 15 w/v %, about 1 w/v % to about 15 w/v %, about 3 w/v % to about 15 w/v %, about 3 w/v %, about 5 w/v %, about 7.5 w/v %, about 10 w/v %, about 12.5 w/v %, or about 15 w/v %.

Also provided herein are nanofibrous scaffolds. The nanofibrous scaffolds disclosed herein can comprise a biodegradable copolymer of the disclosure, a polyspirolactide, or a combination thereof. The nanofibrous scaffold can be used in methods of regenerating tissue. The methods of regenerating tissue can comprise implanting the nanofibrous scaffold of the disclosure in a tissue.

Many modifications and other embodiments disclosed herein will come to mind to one skilled in the art to which the disclosed compounds and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

It is also to be understood that the terminology used herein is to describe particular aspects only and is not intended to be limiting. As used in the specification and the claims, the term “comprising” can include the aspects of “consisting essentially of” and “consisting of.” Unless defined otherwise, all technical and scientific terms used herein have the same meaning as commonly understood by one of ordinary skill in the art to which the disclosed compositions and methods belong. In this specification and in the claims which follow, reference will be made to several terms which shall be defined herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events described or in any other order that is logically possible.

### Definitions

As used herein, the term “alkyl” refers to straight chained and branched saturated hydrocarbon groups containing one to twenty-two carbon atoms, for example, one to twenty carbon atoms, or one to ten carbon atoms. The term Oₙ means the alkyl group has “n” carbon atoms. For example, C₄ alkyl refers to an alkyl group that has 4 carbon atoms. C₁₋₂₂ alkyl and C₁-C₂₂ alkyl refer to an alkyl group having a number of carbon atoms encompassing the entire range (i.e., 1 to 22 carbon atoms), as well as all subgroups (e.g., 1-20, 2-15, 1-5, 3-10, 1, 2, 3, 4, 5, 6, 7, 8, 9, 10, 11, 12, 13, 14, 15, 16, 17, 18, 19, 20, 21, and 22 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group. For example, a “haloalkyl” is an alkyl group that is substituted with one or more halo atoms, and can be perhalogenated (i.e., each hydrogen atom of the alkyl group is substituted with a halo atom).

As used herein, the term “cycloalkyl” refers to an aliphatic monocyclic or polycyclic hydrocarbon ring containing five to twelve carbon atoms, for example, five to ten, five to eight carbon atoms, or five to seven carbon atoms (e.g., 5, 6, 7, 8 carbon atoms). The term Cn means the cycloalkyl group has “n” carbon atoms. For example, C₅ cycloalkyl refers to a cycloalkyl group that has 5 carbon atoms in the ring. C₅₋₅ cycloalkyl and C₅-C₈ cycloalkyl refer to cycloalkyl groups having a number of carbon atoms encompassing the entire range (i.e., 5 to 8 carbon atoms), as well as all subgroups (e.g., 5-6, 6-7, 6-8, 7-8, 5-7, 5, 6, 7, and 8 carbon atoms). Nonlimiting examples of cycloalkyl groups include cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated or fused to another cycloalkyl group, a heterocycloalkyl group, an aryl group and/or a heteroaryl group.

As used herein, the term “heterocycloalkyl” is defined similarly as cycloalkyl, except the ring contains one to four heteroatoms independently selected from oxygen, nitrogen, and sulfur. In particular, the term “heterocycloalkyl” refers to a ring containing a total of five to twenty atoms, for example five to fifteen atoms, five to twelve, or five to ten atoms, of which 1, 2, 3, or 4 of those atoms are heteroatoms independently selected from the group consisting of oxygen, nitrogen, and sulfur, and the remaining atoms in the ring are carbon atoms. Nonlimiting examples of heterocycloalkyl rings include piperdine, pyrazolidine, tetrahydrofuran, tetrahydropyran, dihydrofuran, and morpholine. The heterocycloalkyl groups described herein can be isolated or fused to another heterocycloalkyl group, a cycloalkyl group, an aryl group, and/or a heteroaryl group. In some embodiments, the heterocycloalkyl groups described herein comprise one oxygen ring atom (e.g., oxiranyl, oxetanyl, tetrahydrofuranyl, and tetrahydropyranyl). The heterocycloalkyl can include one or more unsaturated bonds, but is not aromatic. Unless otherwise indicated, a heterocycloalkyl group can be an unsubstituted or a substituted heterocycloalkyl group.

As used herein, the term “alkenyl” is defined identically as “alkyl,” except for containing at least one carbon-carbon double bond, and having two to thirty carbon atoms, for example, two to twenty carbon atoms, or two to ten carbon atoms. The term Cₙ means the alkenyl group has “n” carbon atoms. For example, C₄ alkenyl refers to an alkenyl group that has 4 carbon atoms. C₂₋₇ alkenyl and C₂-C₇ alkenyl refer to an alkenyl group having a number of carbon atoms encompassing the entire range (i.e., 2 to 7 carbon atoms), as well as all subgroups (e.g., 2-6, 2-5, 3-6, 2, 3, 4, 5, 6, and 7 carbon atoms). Specifically contemplated alkenyl groups include ethenyl, 1-propenyl, 2-propenyl, and butenyl. Unless otherwise indicated, an alkenyl group can be an unsubstituted alkenyl group or a substituted alkenyl group.

As used herein, the term “cycloalkenyl” is defined identically as “cycloalkyl,” except for containing at least one carbon-carbon double bond, and containing three to twenty carbon atoms (e.g., 3, 4, 5, 6, 7, 8, 10, 12, 14, 15, 16, 17, 18, 19 or 20 carbon atoms). The term Cn means the cycloalkenyl group has “n” carbon atoms. For example, C₅ cycloalkenyl refers to a cycloalkenyl group that has 5 carbon atoms in the ring. C₅₋₈ cycloalkenyl refers to cycloalkenyl groups having a number of carbon atoms encompassing the entire range (i.e., 5 to 8 carbon atoms), as well as all subgroups, as previously described for “cycloalkyl.” Unless otherwise indicated, a heterocycloalkyl group can be an unsubstituted or a substituted heterocycloalkyl group.

As used herein, the term “aryl” refers to monocyclic or polycyclic (e.g., fused bicyclic and fused tricyclic) carbocyclic aromatic ring systems. The term On means the aryl ring has “n” carbon atoms. For example, C₆ aryl refers to an aryl ring that has 6 carbon atoms in the ring. Examples of aryl groups include, but are not limited to, phenyl, methoxyphenyl, chlorophenyl, naphthyl, methylnaphthyl, fluoronaphthyl, tetrahydronaphthyl, phenanthrenyl, indanyl, indenyl, anthracenyl, tetracenyl, chrysenyl, triphenylenyl, pyrenyl, fluorenyl. Unless otherwise indicated, an aryl group can be an unsubstituted aryl group or a substituted aryl group. In particular, one to four carbon atoms of an aryl ring can be independently substituted with a group selected from, for example, halo, alkyl, alkenyl, OCF₃, NO₂, CN, NC, OH, alkoxy, amino, CO₂H, CO₂alkyl, aryl, and heteroaryl.

As used herein, the term “heteroaryl” refers to a monocyclic or polycyclic aromatic ring system having five to twenty total ring atoms (e.g., a monocyclic aromatic ring with 5-12 total ring atoms), of which 1, 2, 3, or 4 of those atoms are heteroatoms independently selected from the group consisting of oxygen, nitrogen, and sulfur, and the remaining atoms in the ring are carbon atoms. Unless otherwise indicated, a heteroaryl ring can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. In some cases, the heteroaryl ring is substituted with one or more of alkyl and alkoxy groups. Heteroaryl rings can be isolated (e.g., pyridyl) or fused to another heteroaryl group (e.g., purinyl), a cycloalkyl group (e.g., tetrahydroquinolinyl), a heterocycloalkyl group (e.g., dihydronaphthyridinyl), and/or an aryl group (e.g., benzothiazolyl and quinolyl). Examples of heteroaryl groups include, but are not limited to, thienyl, furyl, pyridyl, pyrrolyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl. When a heteroaryl ring is fused to another heteroaryl group, then each ring can contain five to twenty total ring atoms and one to five heteroatoms in its aromatic ring.

As used herein, the term “cyclic group” refers to any ring structure comprising a cycloalkyl, heterocycloalkyl, aryl, heteroaryl, cycloalkenyl, heterocycloalkenyl, or a combination thereof. Unless otherwise indicated, a cyclic group can be an unsubstituted or a substituted cyclic group. As used herein, the term “bicyclic group” refers to a cyclic group that includes two, three or more rings, which can include heteroatoms, that share at least two bonds and three atoms. The bicyclic groups can be, for example, adamantyl, norbornyl, decalinyl, octahydro-1H-indenyl, bicyclo[2.2.2] octanyl, octahydropentalenyl, and the like. In embodiments, the bicyclic group can include a carbon-carbon double bond functionality.

As used herein, the term “hydroxy” or “hydroxyl” refers to the “—OH” group. As used herein, the term “thiol” refers to the “—SH” group.

As used herein, the term “carboxy” or “carboxyl” refers to a —C(═O)OH group and the term “carboxylate” refers to a —C(═O)O⁻ group. The carboxylate group can be associated with an alkali metal or alkaline earth metal cation.

As used herein, the term “halo” is defined as fluoro, chloro, bromo, and iodo. The term “haloalkyl” refers to an alkyl group that is substituted with at least one halo atom, and includes perhalogenated alkyl (i.e., all hydrogen atoms substituted with halo atoms).

As used herein, the term “amino” refers to a —NH₂ group, wherein one or both hydrogens can be replaced with an alkyl, cycloalkyl, or aryl group. As used herein, the term “amido” refers to —NR^{F}C(═O) or —C(═O)—NR^{F}), wherein R^{F} is a substituent on the nitrogen (e.g., alkyl or H). As used herein “imine” refers to a —N(H)═CH₂ group, wherein one, two, or three hydrogens can be replaced with an alkyl, cycloalkyl, or aryl group. When referring to a ligand, the term “amine” refers to a NH₃ group, where one, two, or three hydrogens can be replaced with an alkyl, cycloalkyl, or aryl group. When referring to a ligand, the term “amide” refers to a NH₂ group, wherein one or both hydrogen can be replaced with an alkyl, cycloalkyl, or aryl group.

As used herein, the term “ester” refers to a —C(═O)—O—R group, wherein the R group is an alkyl, cycloalkyl, aryl, or the like.

As used herein, the term “substituted,” when used to modify a chemical functional group, refers to the replacement of at least one hydrogen radical on the functional group with a substituent. Substituents can include, but are not limited to, alkyl, cycloalkyl, alkenyl, cycloalkenyl, alkynyl, heterocycloalkyl, heterocycloalkenyl, ether, polyether, thioether, polythioether, aryl, heteroaryl, hydroxyl, oxy, alkoxy, heteroalkoxy, aryloxy, heteroaryloxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

When a substituent is indicated to be optionally substituted, that means that one or more of an available hydrogen is replaced with a different moiety.

As used herein, the term “click chemistry reactive pair” refers to a pair of complementary functional groups that is capable of undergoing a “click chemistry” reaction. As used herein, “first functional group of a click chemistry reactive pair” refers to one of the pair of complementary functional groups that is capable of undergoing a “click chemistry” reaction. In general, there are four main classes of click chemistry reactions: 1) cycloadditions, 2) nucleophilic ring-openings, 3) carbonyl chemistry of the non-aldol type, and 4) additions to carbon-carbon multiple bonds. The click chemistry reactive pair can be a pair of complementary functional groups that are compatible with the four classes of click chemistry reactions shown above, such as thiol/alkene, azide/alkynes, azide/alkene, alkene/tetrazine, isonitrile/tetrazine, etc. Further examples of click chemistry reactive pairs can be found in Wang et. al., Pharm Res., 2008, 25(10): 2216-2230; Bowman et al., Adv. Fund. Mater., 2014, 24, 2572-2590; and Jozwiak et al., Chem. Rev., 2013, 113, 4905-4979.

As used herein, the term “biodegradable”, “biodegradation”, or “biodegrade” refers to the degradation of a polymer into its component subunits, or digestion, e.g., by a biochemical process, of the polymer into smaller, non-polymeric subunits. In certain embodiments, biodegradation may occur by enzymatic mediation, degradation in the presence of water (hydrolysis) and/or other chemical species in the body, or both.

As used herein, the term “lactone” refers to an organic compound containing an ester group as part of a cyclic group. In embodiments, the lactone as described herein can have one or more ester groups as part of the cyclic group, such as 2 ester groups in the cyclic group. Derivatives thereof, when referring to lactones, refers to the one or more ester containing cyclic group(s) being substituted with one or more substituents as defined here.

The use of the terms “a,” “an,” “the,” and similar referents in the context of the disclosure herein (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure herein and is not a limitation on the scope of the disclosure herein unless otherwise indicated. No language in the specification should be construed as indicating any non-claimed element as essential to the practice of the disclosure herein.

### Biodegradable Copolymers

The biodegradable copolymers disclosed herein can comprise a structure of:

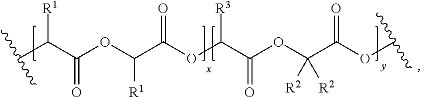

wherein each of x and y is an integer; with a proviso that when the geminal R² groups as a pair, together form

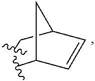 [SMILES: *CC1C=CC(*)C1]

each occurrence of R¹ are C₁-alkyl, and R³ is C₁-alkyl, then the weight average molecular weight of the biodegradable copolymer is about 35 kDa or more. Although the x and y segments are depicted in a block copolymer configuration, the depiction is not intended to be limiting; rather, the x and y segments can be provided in a block copolymer configuration, alternating copolymer configuration, or random copolymer configuration. In embodiments, the biodegradable copolymer has a random configuration of the x and y segments. In embodiments, the biodegradable copolymer has an alternating configuration of the x and y segments. In embodiments, the biodegradable copolymer has a block configuration of x and y segments.

In general, each R¹ is independently selected from C₁-C₂₂ alkyl, C₂-C₂₂ alkenyl, C₅-C₁₂ cycloalkyl, Ar¹, carboxyl, ester, amino, thiol, halo, C₁-C₂₂ haloalkyl, SH, OH, or a first functional group of a click chemistry reactive pair. In embodiments, at least one R¹ is C₁-C₂₂ alkyl. In embodiments, each R¹ is C₁-C₂₂ alkyl. In embodiments, at least one R¹ is C₁-C₆. In embodiments, each R¹ is C₁-C₆. In embodiments, at least one R¹ is methyl, ethyl, n-propyl, isopropyl, n-butyl, sec-butyl, t-butyl. In embodiments, at least one R¹ is methyl. In embodiments, each R¹ is methyl. In embodiments, at least one R¹ is a first functional group of a click chemistry reactive pair, such as a C₂-C₂₂ alkenyl. In embodiments, at least one R¹ is C₂-C₂₂ alkenyl. In embodiments, at least one R¹ is C₂-C₅ alkenyl.

In general, the geminal R² groups as a pair, together with the carbon atom to which they are attached, form a five- to twelve-member cyclic or bicyclic group, or each R² is independently selected from H, C₂-C₂₂ alkenyl, C₅-C₈cycloalkenyl, carboxyl, amido, C₁-C₂₂ haloalkenyl, OH, SH, and Ar¹, or a first functional group of a click chemistry reactive pair. In embodiments, at least one R² is H, C₂-C₂₂ alkenyl, C₅-C₈cycloalkenyl, or SH. In embodiments, at least one R² is a first functional group of a click chemistry reactive pair, such as C₂-C₂₂ alkenyl or SH. In embodiments, the geminal R² groups as a pair, together with the carbon atom to which they are attached, form a five- to twelve-member cyclic or bicyclic group. In embodiments, the geminal R² groups as a pair, together form

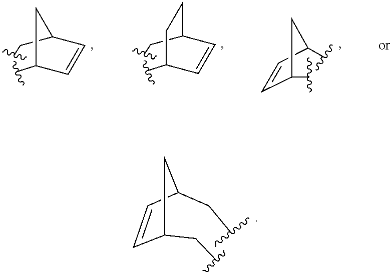 [SMILES: *CC1C=CC(*)C1 ; *CC1C=CC(*)CC1 ; *C1C=CC(*)C1 ; *CC1C=CC(C*)C1]

In embodiments, the geminal R² groups as a pair, together form

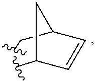 [SMILES: *CC1C=CC(*)C1]

In embodiments, the geminal R² groups as a pair, together with the carbon atom to which they are attached, form a first functional group of a click chemistry reactive pair, such as tetrazine.

In general, R³ is selected from C₁-C₂₂ alkyl, C₂-C₂₂ alkenyl, C₅-C₁₂cycloalkyl, C₅-C₁₂ cycloalkenyl, Ar¹, carboxyl, ester, amino, thiol, halo, C₁-C₂₂ haloalkyl, SH, OH, or a first functional group of a click chemistry reactive pair. In embodiments, R³ is C₁-C₂₂ alkyl. In embodiments, R³ is C₁-C₆. In embodiments, R³ is methyl, ethyl, n-propyl, isopropyl, n-butyl, sec-butyl, t-butyl. In embodiments, R³ is methyl. In embodiments, R³ is a first functional group of a click chemistry reactive pair, such as a C₂-C₂₂ alkenyl. In embodiments, R³ is C₂-C₂₂ alkenyl. In embodiments, R³ is C₂-C₅ alkenyl.

In general, each Ar¹ is independently selected from C₆-C₂₂ aryl or a 5-12 membered heteroaryl comprising from 1 to 3 ring heteroatoms selected from O, N, and S. In embodiments, at least one Ar¹ is phenyl, tolyl, methoxyphenyl, chlorophenyl, naphthyl, methylnaphthyl, or anthracenyl. In embodiments, at least one Ar¹ is thienyl, furyl, pyridyl, pyrrolyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, or thiadiazolyl. In embodiments, at least one Ar¹ is phenyl or tolyl.

In general, each of x and y can be an integer. It will be understood that the absolute values for each of x and y describe the degree of polymerization of the copolymer. Thus the actual values of x and y are not particularly limited, provided when the geminal R² groups as a pair, together form

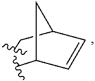 [SMILES: *CC1C=CC(*)C1]

each occurrence of R₁ are C₁-alkyl, and R₃ is C₁-alkyl, then the weight average molecular weight of the biodegradable copolymer is about 35 kDa or more. In embodiments, each of x and y can be an integer in a range of about 1 to about 1000. In embodiments, the ratio of total y segments to total x segments (y:x), can be about 1:100 to about 1:1, or 1:25 to about 1:1 or about 1:10 to about 1:1.1n embodiments, the ratio of y:x can be about 1:100 to about 100:1, or about 1:25 to about 25:1, 1:10 to about 10:1, or about 1:10 to about 2:1, or about 1:10 to about 1:1, or about 1:8 to about 1:1, or about 1:5 to about 1:2, or about 1:2 to about 1:4. For example, the ratio of y:x can be about 10:1, 5:1, 3:1, 2:1, 1:1, 1:2, 1:3, 1:4, 1:5, 1:6, 1:7, 1:8, 1:9, or 1:10. It will be understood that the biodegradable copolymer described herein can be any type of copolymer, i.e., block copolymer, random copolymer, or alternating copolymer. In embodiments, the ratio of x and y (y:x) can be 1:1 and the copolymer can be a perfectly alternating copolymer. In embodiments, the ratio of x and y can be 1:1 and the copolymer can be an alternating polymer (for example, when each of x and y are 3). In embodiments, x and y can be any integer and the copolymer is a random copolymer. In embodiments, x and y can be any integer and the copolymer can be a block copolymer. It is also contemplated that the x monomer unit and/or the y monomer unit is present twice, for example in an x-y-x or y-x-y, triblock configuration.

In general, the weight average molecular weight of the biodegradable copolymer is not particularly limited. For example, the weight average molecular weight of the biodegradable copolymer can be about 1 kDa or more, or about 5 kDa or more, or about 10 kDa or more, or about 15 kDa or more, or about 20 kDa or more, or about 25 kDa or more, or about 35 kDa or more. In embodiments, the weight average molecular weight of the biodegradable copolymer can be about 1 kDa to about 2000 kDa, or about 5 kDa to about 2000 kDa, or about 5 kDa to about 1500 kDa, or about 5 kDa to about 1250 kDa, or about 5 kDa to about 1000 kDa, or about 10 kDa to about 2000 kDa, or about 20 kDa to 2000 kDa, or about 30 kDa to 2000 kDa, or about 35 kDa to about 2000 kDa, or about 35 kDa to about 1500 kDa, or about 35 kDa to about 1250 kDa, or about 35 kDa to about 1000 kDa, or about 50 kDa to about 2000 kDa, or about 50 kDa to about 1500 kDa, or about 50 kDa to about 1000 kDa, or about 100 kDa to about 1500 kDa, or about 100 kDa to about 1000 kDa, or about 100 kDa to about 500 kDa, or about 100 kDa to about 350 kDa, or about 150 kDa to about 1000 kDa, or about 150 kDa to about 500 kDa, or about 150 kDa to about 350 kDa. In embodiments, the weight average molecular weight of the biodegradable copolymer is 5 kDa or more. In embodiments, the weight average molecular weight of the biodegradable copolymer is 35 kDa or more. In embodiments, wherein the geminal R² groups as a pair, together form

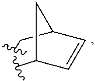 [SMILES: *CC1C=CC(*)C1]

each occurrence of R₁ are C₁-alkyl, and R₃ is C₁-alkyl, then the weight average molecular weight of the biodegradable copolymer is about 35 kDa or more. In embodiments, the weight average molecular weight of the biodegradable copolymer can be about 35 kDa, about 50 kDa, about 75 kDa, about 100 kDa, about 125 kDa, about 150 kDa, about 175 kDa, about 200 kDa, about 250 kDa, about 300 kDa, about 350 kDa, about 400 kDa, about 500 kDa, about 750 kDa, about 1000 kDa, about 1250 kDa, about 1500 kDa, or about 2000 kDa. It will be understood that for biodegradable copolymers having a polydispersity index of greater than 1, and thus a distribution of molecular weights, the weight average molecular weight refers to the mean molecular weight of the distribution.

In general, the biodegradable copolymer as described herein can have a polydispersity index of about 1 to about 2.5. In embodiments, the biodegradable copolymers herein can advantageously have a polydispersity index of about 1 to about 2.2, or about 1 to about 2, or about 1 to about 1.8, or about 1.2 to about 2, or about 1.2 to about 2. For example, the biodegradable copolymers herein can have a polydispersity index of about 1, about 1.1, about, 1.2, about 1.3, about 1.4, about 1.5, about 1.8, about 2, or about 2.2. Polydispersity index is determined according to GPC (Mw) methods as is well known in the art. For example, a polymer sample can be dissolved in a suitable solvent, such as THF, and a Shimadzu GPC or equivalent can be used with suitable software such as Shimadzu-LC LabSolutions or equivalent, to analyze the Mw, Mn, and PDI of the polymers used herein.

In the biodegradable copolymer of the disclosure, none of R¹, R², and R³ form a carbon-carbon double bond with the polymer backbone. Without intending to be bound by theory, it is thought that such a carbon-carbon double bond between a R¹, R², or R³ and the polymer backbone has (a) too much steric hindrance to complete the polymerization and/or (b) it is possible that the extra electron density associated with a carbon-carbon double bond near the polymer backbone can hinder the reactivity of the monomers. As such, for the polymerization process to occur, any carbon-carbon double bond must be at least one carbon removed from the polymer backbone for an efficient polymerization process obtaining high molecular weight copolymers as described herein.

In embodiments, each R¹ is methyl, the geminal R² as a pair, together form

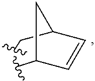 [SMILES: *CC1C=CC(*)C1]

R³ is methyl, and the ratio of y:x is about 1:2 to about 1:4. In embodiments, each R¹ is methyl, the geminal R² as a pair, together form

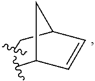 [SMILES: *CC1C=CC(*)C1]

R³ is methyl, and the ratio of y:x is about 1:2 to about 1:4, and the weight average molecular weight is about 100 kDa to about 350 kDa.

### Methods of Lactone Polymerization

The disclosure further provides a method for lactone polymerizations, such as for preparing the biodegradable copolymers of the disclosure. The method advantageously can also be used to prepare homopolymers. The method can comprise two or more steps, the first step (step (a)) comprising: cooling a mixture of monomers to a temperature of about −30° C. to about −110° C. to form a cooled mixture; and the second step (step (b)) comprising: admixing the cooled mixture with about 0.01 mol % to about 5 mol % of a guanidine derivative, based on the total mols of the mixture of monomers, to form the lactone polymer, wherein the mixture of step (b) has a concentration of less than about 10 w/v %. In embodiments, the first step or the second step of the method described herein can further comprise a solvent. In embodiments, the mixture of monomers, the guanidine derivative, or both can comprise a solvent.

The methods of lactone polymerization as disclosed herein have been found to be advantageous as the polymers formed, such as the biodegradable copolymers disclosed herein, can be defined polymers and optionally, defined polymers with high weight average molecular weights (e.g., 35 kDa or more, 50 kDa or more, or 100 kDa or more). As used herein, “defined polymers” refer to polymers having a specific molecular weight (within about 5 kDa) of the target molecular weight and having a low PDI (e.g., less than 2, or less than 1.8). The methods can advantageously be tuned to provide a specified weight average molecular weight by adjusting one or more parameters of the method, such as temperature, monomer concentration, viscosity, catalyst loading, and/or initiator loading. For example, for a given monomer concentration, reaction viscosity, and catalyst/initiator loading, a higher molecular weight polymer can be achieved by lowering the temperature of the reaction (e.g., −80° C.) and a relatively lower molecular weight polymer can be achieved by using a warmer temperature (e.g., −50° C.).

In general, the mixture of monomers can comprise at least one lactone or derivative thereof. In embodiments, the mixture of monomer comprises a first lactone or derivative thereof. As used herein, a lactone or derivative thereof can be any lactone suitable to one of ordinary skill in the art. In embodiments, the first lactone or derivative thereof can be selected from the group consisting of

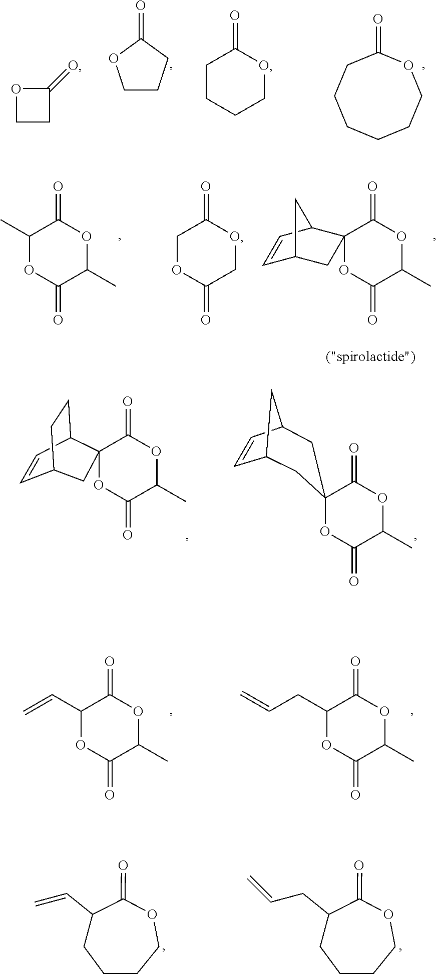

and combinations thereof.

In embodiments, the mixture of monomers can further comprise a second lactone or a derivative thereof. In embodiments, the first lactone can be selected from the group consisting of

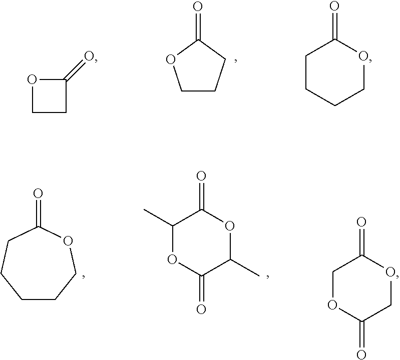

and combinations thereof. In embodiments, the second lactone can be selected from the group consisting of

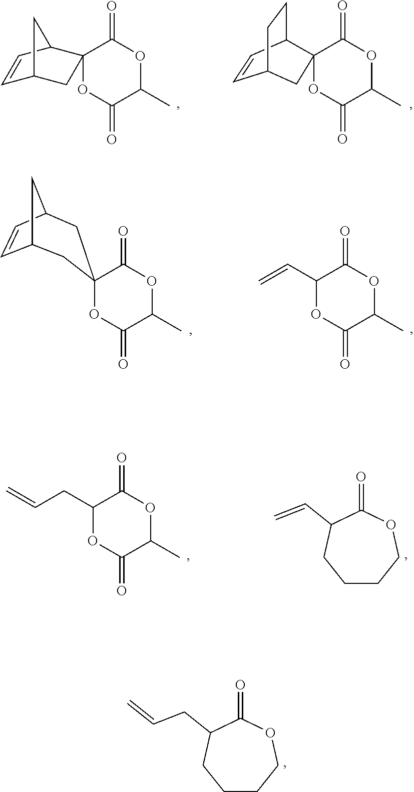

and combinations thereof. In embodiments, the first lactone can comprise

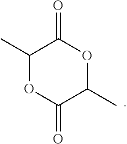

In embodiments, the first lactone can comprise

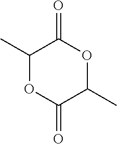

and is the sole lactone in the monomer mixture. In embodiments, the first lactone can comprise

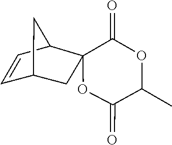

and is the sole lactone in the monomer mixture. In embodiments, the second lactone can comprise

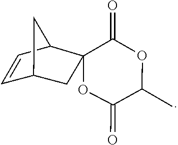

In embodiments, the first lactone can comprise

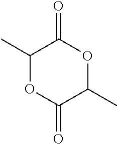

and the second lactone can comprise

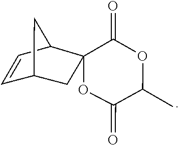

In embodiments, the first lactone or derivative thereof and the second lactone or derivative thereof can be provided in a molar ratio of about 1:1 to about 100:1, or about 1:1 to about 50:1, or about 1:1 to about 25:1, or about 1:1 to about 10:1, or about 1:1 to about 5:1, or about 2:1 to about 10:1, or about 2:1 to about 5:1, or about 2:1 to about 4:1. For example, the first lactone or derivative thereof and the second lactone or derivative thereof are provided in a ratio of about 1:1, 2:1, 3:1, 4:1, 5:1, 8:1, 10:1, 25:1, 50:1, or 100:1. In embodiments, the first lactone or derivative thereof and the second lactone or derivative thereof are provided in a ratio of about 3:1.

The method of preparing a lactone polymer can include cooling a mixture of monomers to a temperature of about −30° C. or less to form a cooled mixture. In embodiments, the mixture of monomers can be cooled to a temperature of about −30° C. to about −200° C., or about −30° C. to about −160° C., −30° C. to about −110° C., or about −30° C. to about −100° C., or about −30° C. to about −90° C., or about −30° C. to about −80° C., or about −50° C. to about −100° C., or about −50° C. to about −80° C. For example, the temperature can be about −30° C., about −40° C., about −50° C., about −60° C., about −70° C., about −80° C., about −90° C., about −100° C., about −110° C., about −120° C., about −130° C., about −150° C., about −160° C., about −200° C. The temperature of the cooled mixture is not particularly limited and can be any temperature lower than −30° C. provided the monomer(s) in the mixture does not precipitate out of the solvent at the temperature of the cooled mixture, the guanidine derivative is at least partially soluble in the solvent at the temperature of the cooled mixture, and the growing polymer chain is at least partially soluble in the solvent at the temperature of the cooled mixture. In embodiments, the mixture of monomers and solvent can be cooled, for example in a cooling bath, for any amount of time suitable to cool the mixture of monomers and solvent to the desired temperature, such as about 10 minutes or more. For example, the mixture of monomers can be cooled for about 10 minutes, 15 minutes, 20 minutes, 25 minutes, 30 minutes, 1 hour, 2 hours, or more. Cooling baths for cooling mixtures to temperatures of about −30° C. to −160° C. are well known in the art.

The method of preparing a lactone polymer comprises admixing the cooled mixture with a guanidine derivative. In embodiments, the guanidine derivative can be represented by a structure of Formula (I):

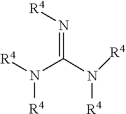

or a salt thereof, wherein each R⁴, independently, is H, C₁-C₁₀ alkyl, C₅-C₈cycloalkyl, carboxyl, ester, amino, thiol, halo, C₁-C₂₂ haloalkyl, OH, and Ar² or two R⁴ groups, together with the atoms to which they are attached, form a five- to eight-member cyclic group, wherein each Ar² is independently selected from C₆-C₂₂ aryl or a 5-12 membered heteroaryl comprising from 1 to 3 ring heteroatoms selected from O, N, and S. In embodiments, the guanidine derivative can be selected from the group consisting of

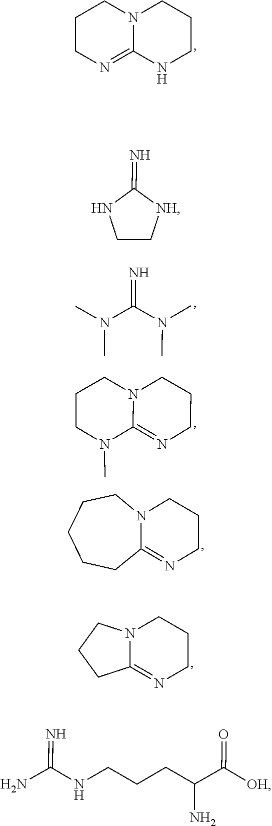

a combination thereof, and salts thereof.

In general, the guanidine derivative has a pKa in the solvent of the cooled mixture. Without intending to be bound by theory, it is believed that as the pKa of the guanidine derivatives increases, a higher molecular weight for the resulting polymer can be accessed. It was found that a guanidine derivative with a relatively high pKa such as, 1,5,7-triazabicyclo[4.4.0]dec-5-ene (TBD) or 7-Methyl-1,5,7-triazabicyclo[4.4.0]dec-5-ene (MTBD), produced lactone polymers that were higher in weight average molecular weights (e.g., 200 kDa or more) with low polydispersity indexes (e.g., 1.8 or less), relative to guanidine derivatives with lower pKas such as, 1,5-Diazabicyclo[4.3.0]non-5-ene (DBN) or 1,8-Diazabicyclo[5.4.0]undec-7-ene (DBU), which showed either lower weight average molecular weights (e.g., less than 150 kDa) or higher polydispersity indexes (e.g., 2 or more), when all other conditions (e.g., monomers, solvent, etc.) were held constant.

In embodiments, the guanidine derivative can be present in an amount of about 0.01 mol % to about 10 mol %, or about 0.01 mol % to about 5 mol %, or about 0.01 mol % to about 3 mol %, or about 0.01 mol % to about 2 mol %, or about 0.01 mol % to about 1 mol %, or about 0.05 mol % to about 0.2 mol %, or about 0.01 mol % to about 0.2 mol %, based on the total mols of the mixture of monomers. For example, the guanidine derivative can be present in an amount of about 0.01 mol %, 0.05 mol %, 0.1 mol %, 0.2 mol %, 0.3 mol %, 0.5 mol %, 1 mol %, 2 mol %, 3 mol %, 5 mol %, or 10 mol %, based on the total mols of the mixture of monomers.

The admixing of the cooled mixture with the guanidine derivative can be for about 5 minutes to about 72 hours, about 10 minutes to about 48 hours, about 15 minutes to about 24 hours, about 30 minutes to about 24 hours, about 45 minutes to about 24 hours, about 1 hour to about 24 hours, or about 5 hours to about 24 hours, or about 12 hours to about 24 hours, or about 18 hours to about 24 hours. For example, admixing the cooled mixture with a guanidine derivative can be for about 1 hour, 5 hours, 10 hours, 12 hours, 15 hours, 18 hours, 20 hours, or 24 hours. In embodiments, the admixing can be in a range of 1 hour to 24 hours. As the length of mixing decreases, the percent conversion of the monomer mixture to the polymer generally decreases. As the length of mixing increases, the percent conversion of the monomer mixture to the polymer generally increases and the molecular weight of the resulting polymer increases. As the length of mixing increases above about 24 hours, additional polymerization may occur, however the molecular weight increase after 24 hours is typically less than 5%, less than 3%, or less than 1% of the weight average molecular weight. The temperature of the cooled mixture, during the admixing with the guanidine derivative, is maintained within ±5° C.

In embodiments wherein the mixture of monomers includes at least a first lactone monomer and a second lactone monomer, the first lactone monomer and second lactone monomer can be provided as a mixture and admixed with the guanidine derivative concurrently. In such embodiments, the resulting copolymer can be a random copolymer. In embodiments wherein the mixture of monomers includes at least a first lactone monomer and a second lactone monomer, the first lactone monomer and second lactone monomer can be admixed with the guanidine derivative in a step-wise manner. In such embodiments, the resulting copolymer can be a block copolymer.

In embodiments, the mixture of monomers is provided in a solvent. In embodiments, the guanidine derivative can be provided in a solvent. When the mixture of monomers and guanidine derivative are provided in a solvent, the solvent of the mixture of monomers and the solvent of the guanidine derivative can be the same or different. In embodiments, the solvent of the mixture of monomers and the solvent of the guanidine derivative are the same. In embodiments, the solvent of the mixture of monomers and the solvent of the guanidine derivative are different. The solvent of the method herein can comprise any one or more organic solvents suitable to at least partially dissolve the guanidine derivative in the solvent at the temperature of the cooled mixture. As used herein, the term “partially soluble” means that at least 3 mol % of a compound is soluble in the solvent, based on the total mols of the compound. For example, at least 3 mol %, at least 5 mol %, at least 7.5 mol %, at least 10 mol %, at least 15 mol % or at least 20 mol % of the guanidine derivative is soluble in the solvent, based on the total mols of guanidine derivative. In embodiments, the mixture of monomers is substantially soluble in the solvent at the temperature of the cooled mixture. In embodiments, the biodegradable polymer is at least partially soluble in the solvent at the temperature of the cooled mixture. In embodiments, the biodegradable polymer is substantially soluble in the solvent at the temperature of the cooled mixture. As used herein, the term “substantially soluble” refers to the mixture of monomers or the biodegradable polymer being at least 75 mol % soluble in the solvent, based on the total mols of the mixture of monomers or biodegradable polymer, respectively. In embodiments, the solvent can comprise an aprotic solvent. In embodiments, the solvent can comprise dichloromethane (DCM), chloroform, dichloroethylene, tetrahydrofuran, toluene, ethyl acetate, acetone, or a combination thereof.

In embodiments wherein the guanidine derivative is provided in a solvent, the volume of the solvent provided with the guanidine derivate relative to the volume of solvent of the monomer mixture is generally negligible such that the addition of the guanidine derivative and the monomer mixture does not result in an increase in temperature of the cooled mixture.

In general, in step (b), the monomer(s) can have a concentration of about 15 w/v % or less, based on the total volume of solvent. Advantageously, it has been found that keeping the concentration of the monomer(s) in the second step (step (b)) low (i.e., less than 10 w/v %) can facilitate formation of high molecular weight polymers. In embodiments, the mixture of step (b) can have a concentration of about 0.001 w/v % to about 10 w/v %, or about 3 w/v % to about 8 w/v %, or about 0.1 w/v % to about 5 w/v %, or about 0.1 w/v % to about 2 w/v %. For example, the mixture of step (b) can have a concentration of about 0.001 w/v %, or about 0.01 w/v %, or about 0.1 w/v %, or about 1 w/v %, or about 2 w/v %, or about 3 w/v %, or about 5 w/v %, or about 8 w/v %, or about 10 w/v %. As the concentration of monomer(s) in the solvent increases above about 10 w/v %, the viscosity of the reaction increases such that movement of the polymer chains become restricted, thereby inhibiting the ends of the polymer chains from coming into contact with other polymer chain ends, which is needed in order to connect the polymer chains and create high molecular weight polymers. As the concentration of the monomer(s) in the solvent decreases below about 0.01 w/v %, the rate of reaction is slowed significantly or does not occur as the chances of the monomer coming into contact with the catalyst and other monomers decreases significantly. For example, it was found that when the concentration of the monomer(s) in the solvent was about 1 w/v %, it was possible to achieve polymers having weight average molecular weights of about 80 kDa and as the concentration of monomer(s) decreased below about 1 w/v %, the weight average molecular weights similarly decreased.

In embodiments, step (b) can further comprise an initiator. In embodiments, the initiator can comprise propanol amine, hydroxyethylmethacrylate, 2-hydroxyethyl acrylate, benzyl alcohol, propargyl alcohol, propanol bromide, 2,2-bis)bromomethyl)-1,3-propanediol, pentaerythritol, pentaerythritol triacrylate, or a combination thereof. In embodiments, the initiator can comprise compound comprising a hydroxy (—OH), for example, a bifunctional polyethylene glycol, wherein the bifunctional polyethylene glycol comprises a hydroxy (—OH). Advantageously, the compound comprising the hydroxy group can further include a first functional group of a click chemistry reactive pair, such that the compound can both initiate polymerization and provide to the polymer chain a first functional group of a click chemistry pair that can be later functionalized.

During testing of the methods provided herein, it was unexpectedly found that a low temperature (e.g., −30° C. or less) and a low concentration of monomer(s) (e.g. less than 10 w/v %) in step (b) provided advantageous lactone polymers with high molecular weights (i.e., 35 kDa or more) and low polydispersity indexes (i.e., 1.8 or less). The methods provided herein can be tuned to control the weight average molecular weight of the lactone polymer. It has been found that as the temperature of the reaction is lowered for a mixture having a concentration in step (b) in a range of about 5 w/v % to about 10 w/v %, the weight average molecular weight of the lactone polymer increases. For example, when all conditions are constant except the temperature of the reaction, when the temperature of the cooled mixture was −80° C. and the concentration of the monomers in step (b) was 5 w/v %, lactone polymers were formed that had a weight average molecular weight of 350 kDa and when the temperature of the cooled mixture was −50° C. and the concentration of monomers in step (b) was 5 w/v %, lactone polymers were formed that had a weight average molecular weight of 200-225 k Da.

In embodiments, the weight average molecular weight of the lactone polymer, such as the biodegradable copolymer disclosed herein, formed in the method described herein can be about 1 kDa to about 2000 kDa, or about 5 kDa to about 2000 kDa, or about 5 kDa to about 1500 kDa, or about 5 kDa to about 1250 kDa, or about 5 kDa to about 1000 kDa, or about 10 kDa to about 2000 kDa, or about 20 kDa to 2000 kDa, or about 30 kDa to 2000 kDa, or about 35 kDa to about 2000 kDa, or about 35 kDa to about 1500 kDa, or about 35 kDa to about 1250 kDa, or about 35 kDa to about 1000 kDa, or about 50 kDa to about 2000 kDa, or about 50 kDa to about 1500 kDa, or about 50 kDa to about 1000 kDa, or about 100 kDa to about 1500 kDa, or about 100 kDa to about 1000 kDa, or about 100 kDa to about 500 kDa, or about 100 kDa to about 350 kDa, or about 150 kDa to about 1000 kDa, or about 150 kDa to about 500 kDa, or about 150 kDa to about 350 kDa. In embodiments, the weight average molecular weight of the lactone polymer can be about 35 kDa, about 50 kDa, about 75 kDa, about 100 kDa, about 125 kDa, about 150 kDa, about 175 kDa, about 200 kDa, about 250 kDa, about 300 kDa, about 350 kDa, about 400 kDa, about 500 kDa, about 750 kDa, about 1000 kDa, about 1250 kDa, about 1500 kDa, or about 2000 kDa.

The method of the disclosure can further comprise admixing a second mixture comprising monomers and solvent after the admixing of the cooled mixture with the guanidine derivative. In embodiments, the second mixture of monomers can comprise the same or different monomers than the cooled mixture. In embodiments, the cooled mixture includes a first lactone or derivative thereof and a second lactone or a derivative thereof, and the second mixture of monomers comprises the first lactone or derivative thereof. In embodiments, the cooled mixture includes a first lactone or derivative thereof and a second lactone or a derivative thereof, and the second mixture of monomers comprises the second lactone or derivative thereof. In embodiments, the cooled mixture includes a first lactone or derivative thereof and a second lactone or a derivative thereof, and the second mixture of monomers comprises a third lactone or a derivative thereof. In embodiments wherein the method further includes admixing a second mixture comprising monomers and solvent the second mixture comprises monomers and solvent can be (i) cooled to the temperature of the cooled mixture prior to addition and/or (b) added at a rate such that the temperature of the cooled mixture is not increased.

In some embodiments, the method can comprise (a) cooling a first lactone or derivative thereof, a second lactone or derivative thereof, and a solvent to a temperature of about −30° C. to about −110° C. to form a cooled mixture; (b) admixing the cooled mixture with about 0.01 mol % to about 5 mol % of a guanidine derivative, optionally in a solvent, based on the total mols of the monomers, and allowing the cooled mixture to react with the guanidine derivative for about 1 hour to about 24 hours at the temperature of about −30° C. to about −100° C., wherein after admixing, the concentration of monomers is less than about 10 w/v %, based on the total volume of solvent; and (c) admixing a second portion of the first lactone or derivative thereof and/or the second lactone or derivative thereof to the mixture of (b) and allowing the first lactone or derivative thereof and/or the second lactone or derivative thereof to react for about 1 hour to about 24 hours at the temperature of the temperature of about −30° C. to about −100° C., wherein the mixture of (c) has a concentration of monomers of less than out 10 w/v % based on the total volume of solvent, to form a lactone copolymer of the disclosure.

In some embodiments, the method can comprise (a) cooling a first lactone or derivative thereof and a solvent to a temperature of about −30° C. to about −110° C. to form a cooled mixture; (b) admixing the cooled mixture with about 0.01 mol % to about 5 mol % of a guanidine derivative, optionally in a solvent, based on the total mols of the mixture of monomers, and allowing the cooled mixture to react with the guanidine derivative for about 1 hour to about 24 hours at the temperature of about −30° C. to about −100° C., wherein after admixing, the concentration of monomers is less than about 10 w/v %, based on the total volume of solvent; and (c) admixing a second lactone or derivative thereof to a the mixture of (b) and allowing the second lactone or derivative thereof to react for about 1 hour to about 24 hours at the temperature of about −30° C. to about −100° C., wherein the mixture of (c) has a concentration of monomers of less than out 10 w/v % based on the total volume of solvent, to form a lactone copolymer of the disclosure.

In some embodiments, the method can comprise (a) cooling a first lactone or derivative thereof and a solvent to a temperature of about −30° C. to about −110° C. to form a cooled mixture; (b) admixing the cooled mixture with about 0.01 mol % to about 5 mol % of a guanidine derivative, optionally in a solvent, based on the total mols of the mixture of monomers, and allowing the cooled mixture to react with the guanidine derivative for about 1 hour to about 24 hours at the temperature of about −30° C. to about −100° C., wherein after admixing, the concentration of monomers is less than about 10 w/v %, based on the total volume of solvent; and (c) admixing a second lactone or derivative thereof to a the mixture of (b) and allowing the second lactone or derivative thereof to react for about 1 hour to about 24 hours at the temperature of about −30° C. to about −100° C., wherein the mixture of (c) has a monomer concentration of less than about 10 w/v %, based on the total volume of solvent; and (d) admixing second portion of the first lactone or derivative thereof, a second portion of the second lactone or derivative thereof, a third lactone or derivative thereof, or a combination thereof, to the mixture of (c) and allowing reaction for about 1 hour to about 24 hours at the temperature of about −30° C. to about −100° C., wherein the mixture of (d) has a monomer concentration of less than out 10 w/v % based on the total volume of solvent, to form a lactone copolymer of the disclosure.

In embodiments, the methods of preparing the lactone polymer herein occur under an inert atmosphere. As used herein, the term “inert atmosphere” refers to an atmosphere that is substantially free of oxygen. An inert atmosphere can include, for example an atmosphere of inert gases, such as N2 or Ar. As used herein and unless specified otherwise “substantially free of oxygen” refers to an oxygen concentration of 5 ppm or less. For example, the oxygen concentration can have a concentration of less than 5 ppm, less than 4 ppm, less than 3 ppm, less than 2 ppm, less than 1 ppm, less than 0.5 ppm, or less than 0.1 ppm.

### Nanofibrous Scaffolds

The disclosure also provides nanofibrous scaffolds. The nanofibrous scaffolds can comprise the biodegradable copolymer of the disclosure, polyspirolactide (PSLA), or a combination thereof. In embodiments, the nanofibrous scaffolds can further comprise polylactide (PLA), poly(glycolic acid) (PGA), poly(lactide-co-glycolic acid) (PLGA), polyhydroxybutyrate (PHB), poly(hydroxyvalerate) (PHV), poly(hydroxybutyrate-valerate) (PHBV), poly(dioxanone) (PDS), polycaprolactone (PCL), Poly(glycerol-co-sebacate), or a combination thereof. In embodiments, the nanofibrous scaffold can further comprise polylactide, such as poly(L-lactide), poly(D-lactide), poly(DL-lactide), or a combination thereof. In embodiments, the nanofibrous scaffold can further comprise poly(L-lactide).

To address the clinical need for readily available small diameter vascular grafts, nanofibrous scaffolds (e.g., biomimetic tubular scaffolds) were developed for rapid in situ blood vessel regeneration. In embodiments, covalently attaching biomolecules onto the backbone of the polymers of the disclosure via thiol-ene click chemistry can impart desirable functionalities to the nanofibrous scaffolds. In embodiments, heparin can be conjugated on nanofibrous scaffolds in order to prevent thrombosis when implanted in situ. By controlling the amount of covalently attached heparin we were able to modulate the physical properties of the tubular scaffold, resulting in tunable wettability and degradation rate while retaining the porous and nanofibrous morphology. In some embodiments, the nanofibrous scaffolds disclosed herein can be used as effective vascular grafts able to generate small diameter blood vessels.

In embodiments, the nanofibrous scaffold can comprise the biodegradable copolymer of the disclosure and PLA. As used herein In general, as the amount of PLA increases, relative to the amount of biodegradable copolymer, the more ordered the polymer stacking, which can facilitate fiber formation and increased crystallinity, and structural integrity of the nanofibrous scaffolds. Without intending to be bound by theory, it is believed that because the norbornene rings of the biodegradable copolymer are bulky substituents, the presence of the copolymer can disrupt the stacking of the PLA polymer chains and at high copolymer levels ultimately prevent the formation of the nanofibrous structure. Accordingly, it will be understood that the biodegradable copolymer of the disclosure and the PLA can be provided in any relative amounts, provided that (i) the relative amount of biodegradable copolymer is low enough to not interfere with the PLA stacking and fiber formation and (ii) the relative amount of biodegradable copolymer is high enough to achieve desired sufficiently fast degradation rate of the nanofibrous scaffold. Because the nanofibrous scaffold biodegrades by hydrolytic cleavage, the biodegradability of the scaffold can be tailored by altering the hydrophilicity of the nanofibrous scaffold, for example, by functionalizing the PSLA of the nanofibrous scaffold with a hydrophilic group. Increasing the hydrophilicity of the nanofibrous scaffold can increase the rate of biodegradation of the nanofibrous scaffold because the hydrophilic groups facilitate water contact with the scaffold, promoting the hydrolytic cleavage and degradation. In embodiments, the biodegradable copolymer of the disclosure and PLA are present in a molar ratio of about 1:1 to about 1:100 (biodegradable copolymer:PLA). In embodiments, the biodegradable copolymer of the disclosure and PLA can be present in a molar ratio of about 1:5 to about 1:100, about 1:5 to about 1:50 or about 1:5 to about 1:30, or about 1:5 to about 1:20, or about 1:5 to about 1:10. For example, the biodegradable copolymer of the disclosure and PLA can be present in a molar ratio of about 1:5, about 1:6, about 1:7, about 1:8, about 1:9, about 1:10, about 1:12, about 1:15, about 1:20, about 1:25, about 1:30, about 1:40, about 1:50, about 1:75, or about 1:100. In general, when the biodegradable copolymer of the disclosure and PLA are provided in a molar ratio of less than about 1:5, the nanofibrous scaffolds are less crystalline and the fibrous structure is not formed, and when the biodegradable copolymer of the disclosure and PLA are provided in a molar ratio of greater than about 1:100 the hydrophilicity of the resulting nanofibrous scaffold decreases and as such, the biodegradability of the nanofibrous scaffold decreases.

In embodiments, the nanofibrous scaffold can comprise PSLA. In embodiments, the nanofibrous scaffold can comprise PSLA and polylactide (PLA), poly(glycolic acid) (PGA), poly(lactide-co-glycolic acid) (PLGA), polyhydroxybutyrate (PHB), poly(hydroxyvalerate) (PHV), poly(hydroxybutyrate-valerate) (PHBV), poly(dioxanone) (PDS), polycaprolactone (PCL), Poly(glycerol-co-sebacate), or a combination thereof. In embodiments, the nanofibrous scaffold can comprise PSLA and PLA, such as poly(L-lactide) (PLLA). In embodiments, the PSLA and PLA can be present in a ratio of about 1:8 to about 8:1 or about 1:5 to about 5:1, or about 1:3 to about 3:1, or about 1:2 to about 2:1. For example, the PSLA and PLA can be present in a ratio of about 1:10, about 1:8, about 1:5, about 1:3, about 1:2, about 1:1.5, about 1:1, about 1.5:1, about 2:1, about 3:1, about 5:1, about 8:1 or about 10:1.

The nanofibrous scaffolds as disclosed herein can be molded into any suitable shape to one of ordinary skill in the art. In embodiments, the nanofibrous scaffold is tubular in shape. In some embodiments, the tubular nanofibrous scaffolds can be designed to have an inner layer that is porous, interconnected, and with a nanofibrous architecture, which provided an excellent microenvironment for host cell invasion and proliferation.

The nanofibrous scaffolds disclosed herein can further comprise functionalization selected from the group consisting of anticoagulants, growth factors, hormones, cell-adhesion peptides, receptors, proteins, sugars, lipids, minerals, or a combination thereof. In embodiments, the functionalization can be a growth factor(s). In embodiments, the growth factor can be a vascular endothelial growth factor (VEGF), a platelet derived growth factor (PDGF), fibroblast growth factor (FGF), or a combination thereof. In embodiments, the functionalization can be an anticoagulant compound. In embodiments, the anticoagulant compound comprises heparin, thiolated polyethylene glycol (PEG-SH), zwitterionic poly(carboxybetaine), zwitterionic poly(sulfobetaine), zwitterionic poly(cysteine), zwitterionic poly(phosphatidylcholine), or a combination thereof. In embodiments wherein the nanofibrous scaffold comprises the biodegradable copolymer of the disclosure, the functionalization can be conjugated to said biodegradable copolymer. In embodiments wherein the nanofibrous scaffold comprises PSLA, the functionalization can be conjugated to said PSLA. The functionalization can be present in an amount of about 1 mol % to about 50 mol %, based on the total mols of PSLA monomer units. In embodiments, the functionalization can be present in an amount of about 1 mol % to about 25 mol %, based on the total mols of PSLA monomer units. The functionalization can affect the contact angle of the nanofibrous scaffold. In embodiments, the functionalization can be provided in an amount sufficient to provide a contact angle of about 20° to about 90°. In general, the amount of functionalization sufficient to provide a contact angle between 20° and 90° will depend on the type of functionalization and the contact angle of the material prior to functionalization. In embodiments, the nanofibrous scaffold comprises the biodegradable copolymer of the disclosure and/or PSLA, the functionalization comprises an anticoagulant, and the amount of anticoagulant sufficient to provide a contact angle between 20° and 90° can be about 0.001 mol % to about 100 mol %, based on the total mols of spirolactide monomer. In some embodiments, the amount of anticoagulant sufficient to provide a contact angle between 20° and 90° can be about 0.01 mol % to about 20 mol %, based on the total mols of spirolactide monomer. Without intending to be bound by theory, it is believed that the amount of anticoagulant sufficient to provide a contact angle between 20° and 90° will depend on the size of the specific anticoagulant and the ability of the specific anticoagulant to modify the hydrophilicity of the surface of the nanofibrous scaffold. For example, for a scaffold prepared from 1 g of total polymer (PSLA and PLA), at a 1:1 ratio of PSLA and PLA, and the PSLA comprising 25 mol % spirolactide modification, the scaffold can be functionalized with 10 mg heparin (about 1.66 mol %) to 100 mg heparin (about 16.6 mol %) to provide a contact angle between 20° and 90°. An anticoagulant having a molecular weight less than that of heparin would be expected to require more modification than heparin to achieve the same contact angle due to a smaller impact on the hydrophilicity of the nanofibrous structure surface.

In embodiments, the nanofibrous scaffold has a contact angle of about 20° to about 90°, or about 30° to about 80°, or about 40° to about 70°, or about 50° to about 60°, or about 25° to about 40°. Nanofibrous scaffolds with low contact angles (e.g., 20° to about) 90° can provide advantageous properties. In general, as the contact angle decreases below about 100°, the hydrophilicity of the surface increases and in a range of 20° to about 90° the surface of the nanofibrous scaffolds have a hydrophilicity that advantageously allows the nanofibrous scaffold to demonstrate improved interaction with a surrounding aqueous environment (e.g., biological surrounding), relative to nanofibrous scaffolds having surfaces with contact angles less than about 20° and greater than about 90°.

The nanofibrous scaffolds as disclosed herein can be porous. In embodiments, the nanofibrous scaffolds can have a pore size of about 40 μm to about 600 μm, or about 60 pm to about 500 μm, or about 60 pm to about 450 μm, or about 60 pm to about 400 pm, or about 100 μm to about 400 μm, or about 150 μm to about 400 pm. For example, the pore size can be about 60 pm, about 70 pm, about 80 μm, about 90 pm, about 100 μm, about 150 pm, about 200 μm, about 250 μm, about 300 μm, about 350 μm, about 400 μm, about 450 pm, or about 500 pm. Without intending to be bound by theory, it is believed that as the pore size decreases below about 60 pm, then cells can become trapped on the outer layer of the scaffold as they are seeded, rather than infiltrate the nanofibrous scaffold structure. Advantageously, the pore size can be controlled to provide a nanofibrous scaffold suitable for various intended end uses. For example, wherein the nanofibrous scaffold is intended to be used in cardiovascular tissue, a pore size of about 60 pm to about 150 pm is preferred. For example, wherein the nanofibrous scaffold is intended to be used in bone, a pore size of about 250 μm to about 425 pm is preferred. The pore size of the nanofibrous scaffold can be tailored based on the methods described in Wei, G. and Ma, P. X. (2006), J. Biomed. Mater. Res., 78A: 306-315 and Wang et al., Biomaterials, Volume 35, Issue 32, 2014, pages 8960-8969.]

In embodiments, the nanofibrous scaffold can comprise a bilayer structure. In embodiments, the bilayer structure comprises a porous layer comprising a functionalized biodegradable copolymer and/or PSLA, poly(L-lactic acid), or a combination thereof, and a less porous biodegradable copolymer outer layer. In some embodiments, the bilayer structure comprises a porous layer comprising the functionalized biodegradable copolymer and PLLA, and a less porous biodegradable copolymer outer layer, wherein the functionalized biodegradable copolymer is functionalized with an anticoagulant compound, such as heparin. In embodiments, the nanofibrous scaffold can comprise a bilayer structure and be provided in a tubular form such that the bilayer structure comprises an inner layer comprising a porous layer comprising the functionalized biodegradable copolymer and PLLA, and an outer layer comprises a less porous biodegradable copolymer outer layer.

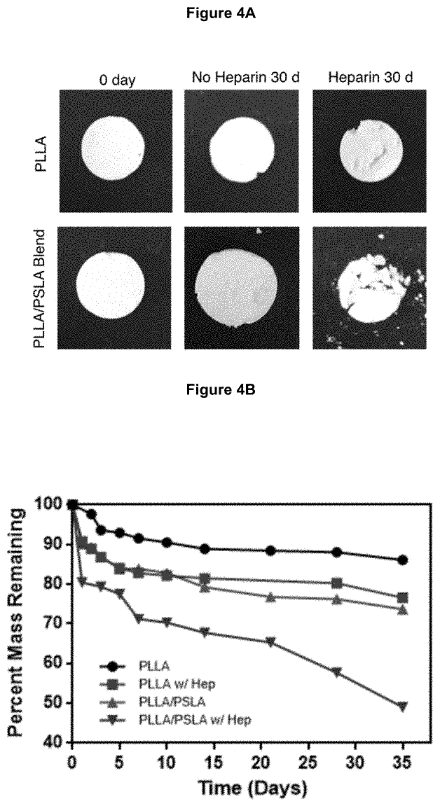

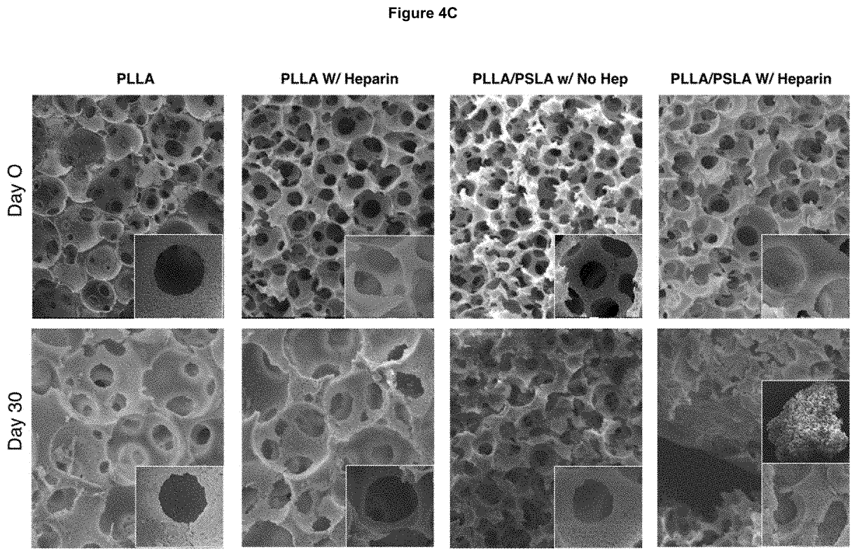

The nanofibrous scaffolds can advantageously degrade in biological environments quickly (e.g., 40% mass loss after about 1 month) compared to other scaffolds (FIG. 4A-4C). In embodiments, the nanofibrous scaffold can have a total mass loss of about 40% or more after storage in phosphate-buffered saline at a temperature of 37° C. for 30 days. In embodiments, the nanofibrous scaffold has a total mass loss of about 50% or more after storage in phosphate-buffered saline at a temperature of 37° C. for 30 days.

In embodiments, the nanofibrous scaffold can be a vascular graft. In embodiments, the nanofibrous scaffold can be used in a spinal cord, cartilage, muscle, bone, or a combination thereof. In embodiments, the nanofibrous scaffold can be used for bone regeneration. In embodiments, the nanofibrous scaffold is cell-free or free of cultured cells. As used herein and unless otherwise indicated, “free of cultured cells” or “cell-free” refers to a nanofibrous scaffold that has not been treated with cultured cells. In embodiments, the nanofibrous scaffold includes cultured cells. In embodiments, the nanofibrous scaffold is cell-free or cell-laden and the nanofibrous scaffold is a vascular graft. In embodiments, the vascular graft is free of cultured cells. Previous methods entail the use of cultured cells to create small-diameter tissue engineered blood vessels for cardiac and peripheral revascularization procedures. Such vascular grafts including cultured cells are not suitable for most clinical applications due to the immediacy of when the vessel is commonly needed. Cell-free tissue engineering vascular grafts, such as the nanofibrous scaffolds disclosed herein, allow for the elimination of the prohibitive lead-time required for cell culture and enable immediate implantation.

Further provided herein is a method of regenerating tissue, the method comprises implanting a nanofibrous scaffold of the disclosure in a tissue or connected to a tissue. Methods of implanting scaffolds in tissue are well known in the art. The method of regenerating tissue disclosed herein can have one or more advantages, including but not limited to, allowing infiltration by native cells into the scaffold in a short period of time (e.g., about a week), providing complete regeneration of the endothelial cells and the smooth muscle cells of a blood vessel, providing a native-like extracellular matrix, and/or tailorable degradation rates of the scaffold, or a combination thereof. In embodiments, it is advantageous to tailor the nanofibrous scaffold as disclosed herein to degrade as the cells move in and deposit the extracellular matrix proteins (e.g., elastin and collagen) such that the cells structure the vessel in a native manner. If the nanofibrous scaffold did not begin to degrade upon infiltration of the cells, there would be insufficient space for the cells to remodel and deposit any further extracellular matrix proteins in the amount needed for a healthy vessel. In embodiments, a functional blood vessel can be regenerated by the body through a nanofibrous scaffold disclosed herein that is biocompatible, anticoagulant, free of cultured cells, and with a tailorable degradation rate. Without intending to be bound by theory, it is believed that, the nanofibrous scaffold can have excellent cellular migration (e.g., natural tissue formation can be seen to begin at 1 month) within the scaffold facilitated by the highly porous and interconnected architecture. Historically, elastin production during in situ vascular tissue engineering has been a major challenge. In embodiments, the methods disclosed herein advantageously can provide large amounts of collagen and elastin being secreted with native-like structure in the engineered blood vessels including the nanofibrous scaffolds. The nanofibrous scaffolds advantageously promote the deposition of large amounts of collagen and elastin by providing pores in sizes that provide an excess of space for cells to deposit the extracellular matrix proteins and through degradation of the scaffold upon infiltration of the cells.

In embodiments, the tissue can comprise fibroblast cells, smooth muscle cells, endothelial cells, or a combination thereof. In embodiments, the nanofibrous scaffold is pre-seeded with cells or infiltrated by host cells after implantation. In embodiments, the nanofibrous scaffold herein can be infiltrated by host cells. In embodiments, the nanofibrous scaffold herein can be infiltrated by host cells in about a week.

Advantageously, the method of regenerating tissue herein can include the implantation of nanofibrous scaffolds of the disclosure that advantageously promote cell migration, attachment, proliferation and smooth muscle regeneration. Advantageously, the method of regenerating tissue herein can be designed to tailor the degradation of the scaffold to the regeneration of tissue by the cells reconstructing it. For example, the degradation rate can be tailored by modifying the hydrophilicity of the nanofibrous scaffold as described above, which can be accomplished in a multitude of ways, including, but not limited to, altering the amount of spirolactide monomer in the PSLA polymer, altering the amount of polyspirolactide polymer in the nanofibrous scaffold, altering the amount of functionalization of the nanofibrous scaffold, or a combination thereof. In particular, for a given cell type, the degradation rate can be controlled such that the degradation of the scaffold is fast enough to allow the cells space to move into the scaffold and reconstruct the tissue, but not so fast as to degrade prior to allowing the cells a chance to occupy and regenerate the tissue environment before the structural support (i.e., nanofibrous scaffold) disappears. The interconnected porous structure of the nanofibrous scaffold can advantageously provide superior cell infiltration and movement, facilitating complete endothelization and formation of a native-like extracellular-matrix at the site of implantation. In addition, advantageously, the method of regenerating tissue can include the functionalized nanofibrous scaffolds of the disclosure herein. In embodiments, wherein the nanofibrous scaffolds are functionalized with an anticoagulant such as heparin, the method of regenerating tissue is advantageous as the hydrophobic polymers of the nanofibrous scaffolds no longer have a negative interaction with the platelets and proteins in the vasculature and thrombosis does not occur. Instead, the anticoagulant, such as heparin, prevents the platelets and proteins from attaching to the nanofibrous scaffold's surface such that thrombosis does not occur. Further, in embodiments wherein the nanofibrous scaffold is functionalized with a hydrophilic functionalization, such as heparin, the methods of regenerating tissue can be provide one or more advantages, such as: 1) creating a positive interaction with the cells which facilitates binding of the cells to the nanofibrous scaffold; 2) creating an environment wherein the scaffold is compatible with the implant environment, allowing water/media/fluid can move through the scaffold without repulsion, facilitating mass transport; or a combination thereof. By combining the advantageous physical structures with the chemical functionalities of the nanofibrous scaffolds, a superior bilayer nanofibrous scaffold can be used to achieve endothelialized blood vessel regeneration with native-like collagen-rich and elastin-rich extracellular matrices. In view of the foregoing advantages, the nanofibrous scaffolds of the disclosure are suitable for implantation in a living being.

### EXAMPLES

### Materials and Methods

L-lactide was donated by Altasorb® and recrystallized in ethyl acetate before use. Poly(L-lactic acid) (PLLA, Resomer L207S) with an inherent viscosity of 1.6 dl/g was purchased from Boehringer Ingelheim (Ingelheim, Germany). Heparin was purchased from Fisher Scientific and modified with L-cysteine. Polyethylene glycol modified at one end with thiol (PEG-SH) of a MW of 10 kDa was purchased from Layson Bio. Polycaprolactone 70-80 k Da (PCL), N-Bromosuccinimide (NBS), triethylamine (TEA), Luperox® A98 benzoyl peroxide, benzene, mineral oil, Span 80®, ethyl acetate, hexane, benzyl alcohol, magnesium sulfate (MgSO₄), sodium thiosulfate, triazabicyclodecene (TBD), L-cysteine, fructose, tetrahydrofuran (THF) and dichloromethane (DCM) were purchased from Sigma-Aldrich Company (USA) and used as received.

### Monomer, Polymer and Scaffold Characterization

Nuclear magnetic resonance characterization: All monomers (L-lactide, exomethylene lactide, and spirolactide) and polymers formed were characterized via 500 MHz ¹H and ¹³C (Varian Inova 500, or equivalent thereof) by dissolving in deuterated chloroform (CDCl₃).

Ultraviolet—visible spectroscopy: All monomers and polymers formed were characterized by first dissolving in dichloromethane (DCM) and using a quartz cuvette to obtain spectrum from 200-700 nm (up to 350 shown) (Hitachi U-2910, or equivalent thereof).

Fourier-transform infrared spectroscopy: Monomers, polymers, and modified tubular scaffolds were characterized by directly placing the sample on a diamond crystal sample holder to obtain the spectrum from 600-4000 nm (Thermo-Nicolet IS-50, or equivalent thereof).

Scanning electron microscopy (SEM) observation: Empty scaffolds were sputter-coated with gold for 150 seconds and observed under a scanning electronic microscope (JEOL JSM-7800FLV, or equivalent thereof).

Gel permeation chromatography: Polymers formed were dissolved in anhydrous tetrahydrofuran and the molecular weight of the polymers were determined by comparing to polystyrene standards (Shimadzu GPC, or equivalent thereof).

### Example 1—Synthesis of a Biodegradable Copolymer

### Spiro[6-methyl-1,4-dioxane-2,5-dione-3,2′-bicyclo[2.2.1]hept[5]ene] synthesis

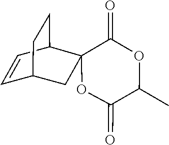 [SMILES: CC1OC(=O)C2(CC3C=CC2CC3)OC1=O]

L-Lactide was modified based on a method previously reported by Hillmyer et al., J Am Chem Soc., 130(42) (2008) 13826-7. Briefly, L-Lactide was combined with n-bromosuccinimide (NBS) (1.1 eq.) in benzene (20% w/v) and heated to reflux. Benzoyl peroxide dissolved in benzene was added dropwise and the reaction was monitored by thin layer chromatography (TLC) until completion. The reaction was vacuum filtrated and condensed and the resulting solid was dissolved in DCM, washed in sodium thiosulfate solution, and concentrated. The solid was recrystallized from ethyl acetate and hexane twice to yield white crystals, (3S, 6S)-3-Bromo-3,6-dimethyl-1,4-dioxane-2,5-dione (bromo-lactide). The white crystals were dissolved in DCM in a flask under nitrogen and cooled in an ice bath. Triethylamine (TEA) (1.1 eq) was added in dropwise and allowed to react for 1 h at 0° C. and 1 h at 25° C. The reaction was washed with 1M HCl three times and condensed. The crystals were further purified by liquid chromatography on silica gel and recrystallization in ethyl acetate to yield white crystals, (6S)-3-Methylene-6-methyl-1,4-dioxane-2,5-dione (methylene lactide). To freshly distilled cyclopentadiene (2.2 eq.) methylene lactide was added (1 eq.) and refluxed overnight in benzene under argon. The reaction was condensed and purified by liquid chromatography in 50/50 hexane:DCM (r_{f}=0.31) followed by recrystallization in ethyl acetate to yield white crystals, Spiro[6-methyl-1,4-dioxane-2,5-dione-3,2′-bicyclo[2.2.1]hept[5]ene] (spirolactide).

### Example 2—General Procedure for the Ring-Opening Polymerization

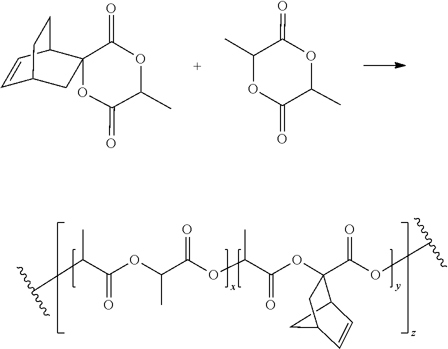

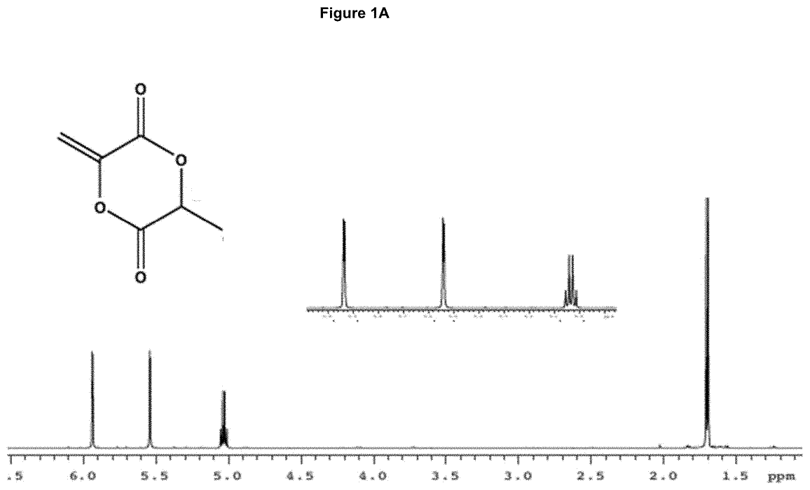

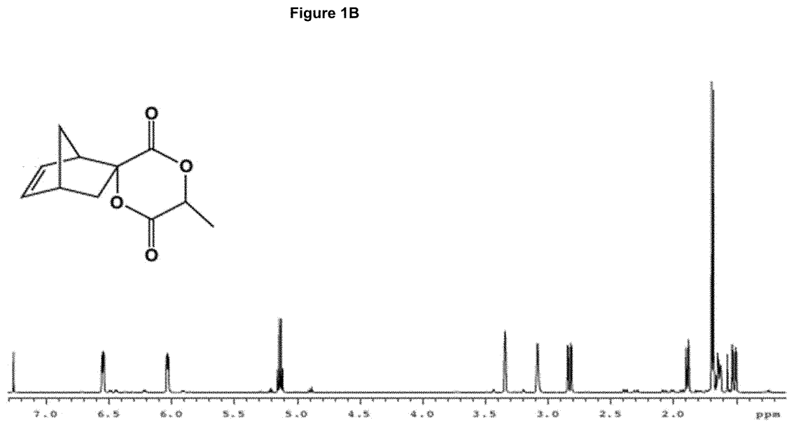

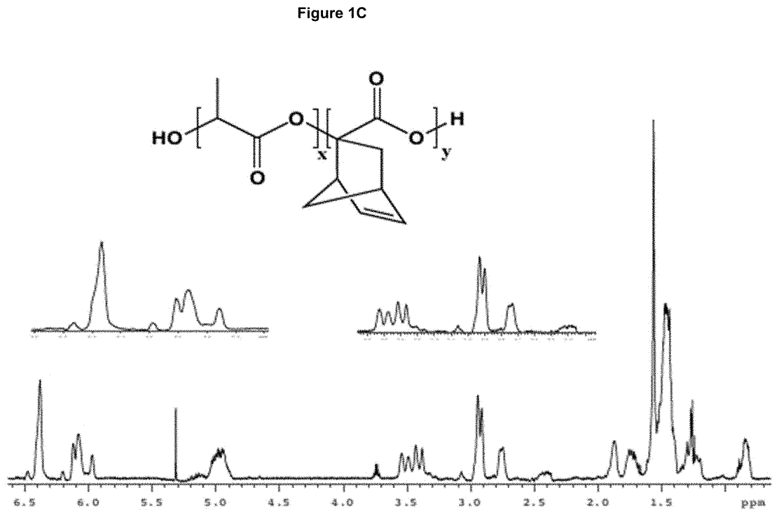

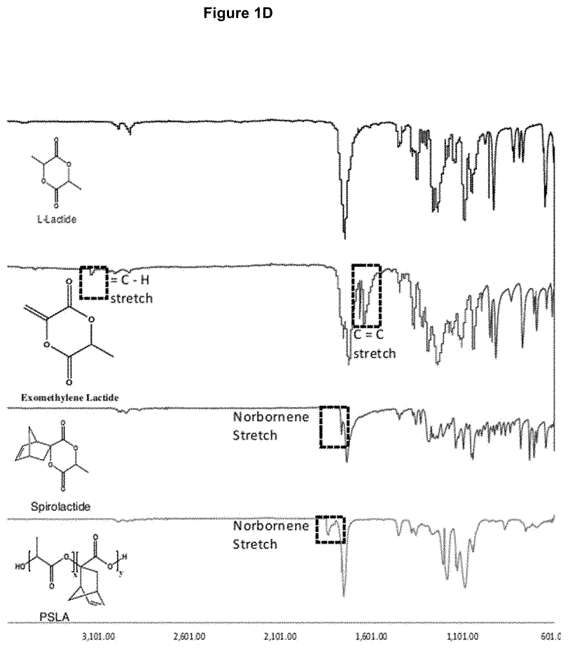

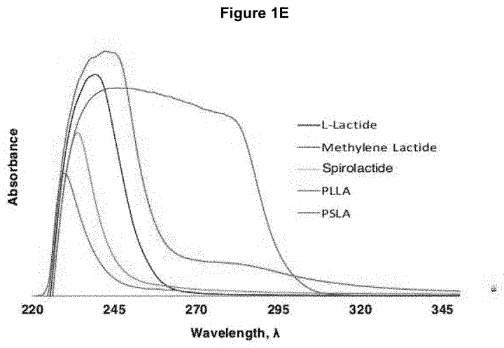

In a reaction flask, the spirolactide from Example 1 was added in a 1:3 molar ratio with L-lactide and the flast was purged three times via a standard vacuum/nitrogen gas treatment before dissolving in anhydrous DCM at 5% w/v. The flask was cooled to −80° C. for 1 h and was then injected with 0.5% mol TBD catalyst dissolved in DCM, such that the concentration of monomers in the solution was maintained at 5% w/v. The reaction was allowed to react at −80° C. for 24 h. The copolymer formed was precipitated in ethyl ether, re-dissolved in DCM and precipitated in ethyl ether, and stored under vacuum. The resulting copolymer was a random copolymer and is referred to herein as poly(spirolactic-co-lactic acid) (PSLA). The monomers and polymer were thoroughly characterized using NMR, UV-VIS, and FTIR (FIG. 1B-1E).

Thus, Example 2 demonstrates the formation of a biodegradable copolymer of the disclosure using the method of the disclosure.

### Example 3—Nanofibrous Tubular Scaffold Fabrication

A tubular scaffold with favorable nanofibrous architecture and tunable physical properties, such as pore size, interconnectivity, internal diameter, and wall thickness was fabricated using a method using a sugar template and thermally induced phase separation (TIPS).

Sugar scaffolds were fabricated in a facile, economic, and reproducible method by using aluminum foil cut to 5 cm wide strips. The aluminum foil was wrapped around a metal rod with the desired external diameter (1.85 mm) and then the metal rod wrapped with the aluminum foil was placed in a premade aluminum mold. Premade fructose sugar spheres were then deposited inside the premade aluminum mold (FIG. 2A-2C) while the premade aluminum mold was submerged in hexane to prevent moisture from agglomerating the fructose sugar spheres, followed by heating to remove the hexanes. The fructose sugar mold was removed from the aluminum mold and aluminum foil. The sugar template spheres were fabricated through an emulsion method developed by Ma et. al, Macromol. Biosci., 12(7) (2012) 911-9 and Adv. Drug Deliver Rev., 60(2) (2008) 184-198. The sugar spheres were separated using sieves to recover spheres ranging from 60-150 μm in diameter. A mandrel with the desired internal diameter (1.0 mm) was placed in the center and the sugar spheres annealed to the desired interconnectivity by placing in a 37° C. incubator for 7 minutes followed by vacuum drying the sugar scaffold.

TIPS formation of the nanofibrous tubular scaffold was carried out according to procedures developed by Ma et al and is described in Ma et al., Wires Nanomed Nanobi 1(2) (2009) 226-236, and Ma et al., J Biomed Mater Res 46(1) (1999) 60-72. The aluminum foil was removed, and the sugar template was dipped into a 10% w/v polymer solution consisting of 50% PLLA (inherent viscosity of 1.6 dl/g) and 50% PSLA, as prepared in Example 2, dissolved in THF, and then quickly cooled to −80° C. in order to allow for phase separation. The polymer scaffolds were maintained at this temperature for 24 h. PCL dissolved in trifluoroethanol (TFE) was electrospun onto the polymer scaffold immediately after brushing the polymer scaffold with a mixture of 90:10 hexanes to THF (15 kV, 10 cm, 2 mL/h, w/rotation). The hexanes:THF mixture created a tacky surface, after brushing, in which the electrospun PCL fibers could anneal to. The polymer scaffolds were then placed in hexanes for 24 h to remove excess THF and TFE and were subsequently placed in double distilled water (DDH2O) to remove the sugar spheres. After the sugar is completely washed away, the mandrel was easily removed, and resulting the nanofibrous polymer scaffold was cut to the desired length.

Thus, Example 3 demonstrates formation of the nanofibrous polymer scaffolds of the disclosure using biodegradable copolymers of the disclosure.

### Example 4—Surface Modification with Anticoagulant Molecules

Tubular nanofibrous polymer scaffolds as prepared according to Example 3 were modified through UV-light induced thiol-ene click-chemistry with the desired molecules to be conjugated. In this example, the molecule to be conjugated is an anticoagulant, heparin. Briefly, methoxy-PEG-SH 2k (50 mg), heparin modified with L-cysteine (100 mg), and tetramethylethylenediamine (TEMED) were dissolved in DDH₂O. The UV-initiator, Irgacure 2959, was dissolved in 100 μL of dimethyl sulfoxide (DMSO) and added to the solution. The polymer scaffolds were quickly soaked in ethanol, added to the PEG/heparin solution, and exposed 340 nm UV-light. The scaffolds were then washed in methanol and DDH₂O to remove excess TEMED and Irgacure 2959 before being lyophilized. The scaffolds were then sterilized with ethylene oxide (Anproline Gas Sterilizer) and stored at −20° C.

Contact Angle Measurements of Thin-Films

Thin-films were fabricated through TIPS processing (as described above) of a 10% polymer solution on a silicon surface to determine the wetness and change of hydrophilicity due to the conjugation of heparin. Films of (a) a PSLA/PLLA blend and (b) only PLLA, were fabricated, and a subfraction of each type were further modified with increasing concentrations of heparin to produce films with the following permeations: PLLA no heparin, PLLA w/ Heparin (50 mg), PSLA/PLLA no heparin, PSLA/PLLA with increasing heparin concentration 10 mg, 50 mg, and 100 mg and a PEG575 gel as a positive control, n=3 each and a standard error of ±2°. The films were attached to a glass side using double sided tape and the advancing contact angle of a drop of water was measured utilizing Rame-Hart 200-F1 contact angle goniometer, or equvalent.

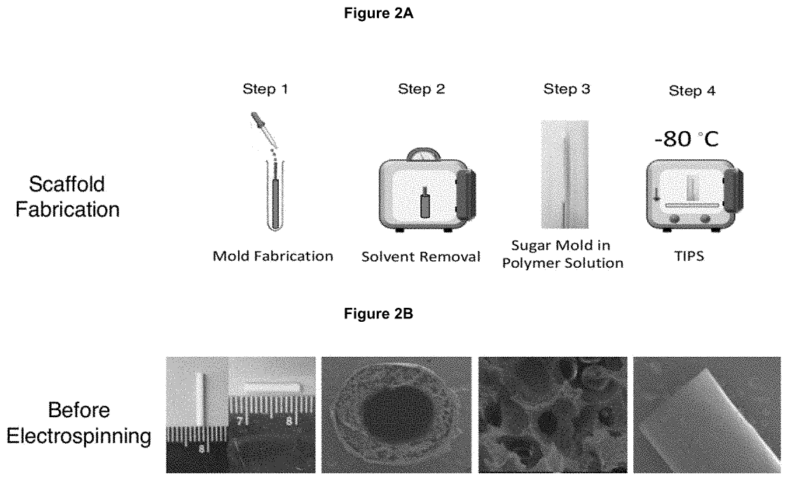

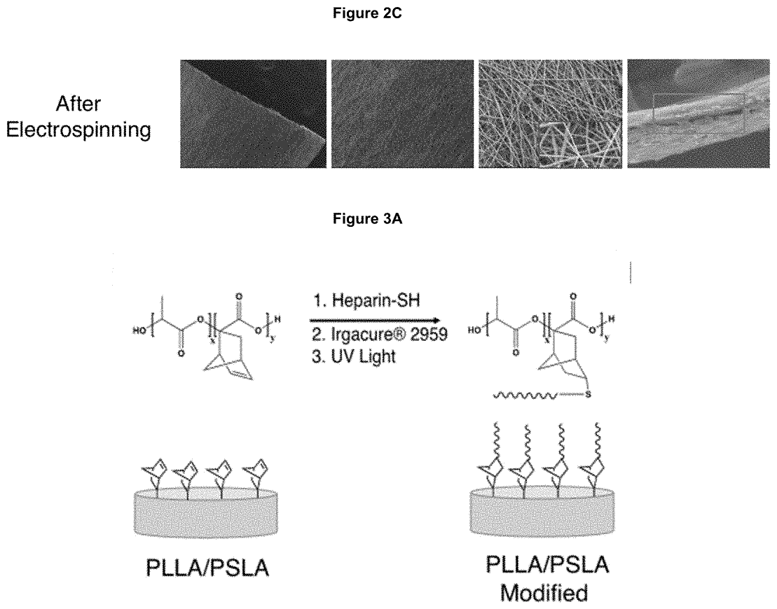

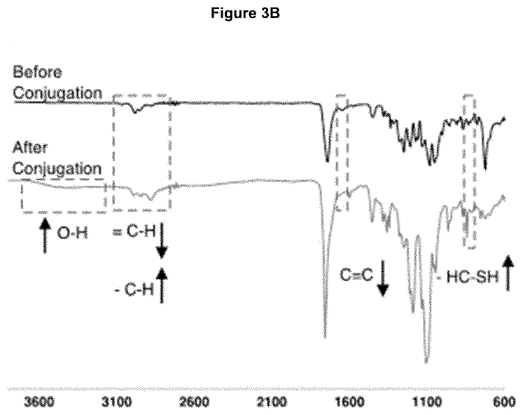

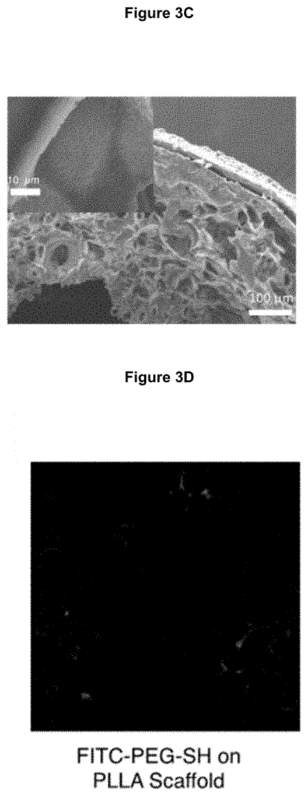

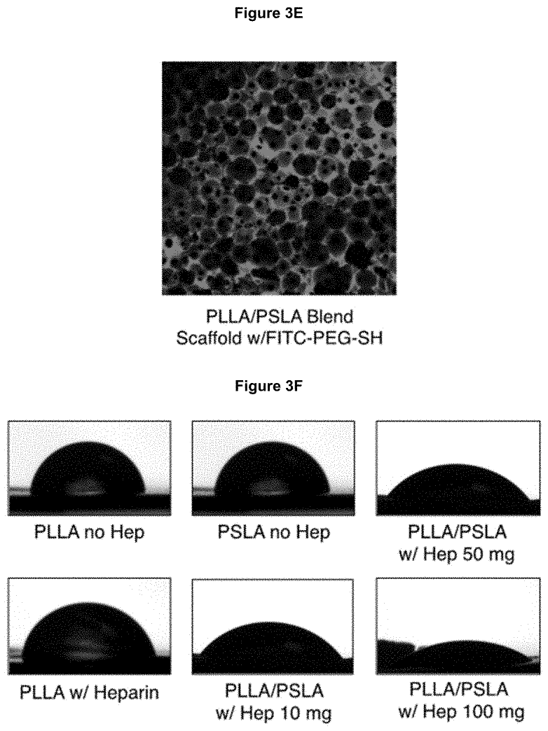

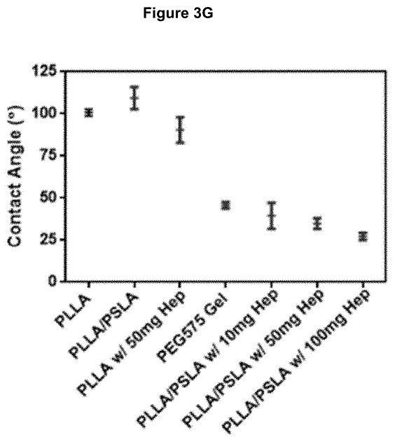

Heparin conjugated films showed increased wettability with contact angles ranging from 25-40° as compared to the unmodified PLLA (100°) and PLLA/PSLA blend (110°) films. This indicates the highly hydrophilic property of the heparin conjugated materials. Both films with 50 mg heparin and with 100 mg heparin were able to absorb the water droplet after 3 and 1 min respectively (FIGS. 3F and 3G).

Thus, Example 4 demonstrates functionalization of the biodegradable copolymers of the disclosure in the form of nanofribrous tubular scaffolds of the disclosure, to provide functionalized biodegradable copolymers and functionalized nanofibrous tubular scaffolds of the disclosure.

### Example 5—“In Vitro” Degradation

Flat three-dimensional scaffolds were fabricated in a Teflon® mold utilizing a sugar template prepared as described above to determine the degradation rate of the polymer of the tubular scaffolds as prepared in Example 3, with or without being modified with heparin. Polymer scaffolds from the PSLA/PLLA blend and PLLA were fabricated and cut into a 2 mm thick disk. As a control, PLLA scaffolds were included with and without heparin conjugated as well as PSLA/PLLA blend scaffolds with and without heparin conjugated, n=5. The polymer scaffolds were placed in a pre-weighed 20 mL vial and then weighed to determine the mass of the scaffold at day 0. To each 20 mL vial containing a scaffold, 10 mL of 1 M PBS pH 7.4 was added and incubated at 37° C. The PBS was removed at predetermined times, the scaffolds were lyophilized, and the mass was taken and recorded. The scaffolds were then submerged in fresh PBS and incubated at 37° C. Images were taken at day 0 and day 35 to track the change in mass loss and degradation. Visually, the PLLA/PSLA group conjugated with heparin began to fall apart at day 10, with all scaffolds breaking apart into large and small pieces by day 28. In comparison, the PLLA group remained intact and only a small amount of scaffold was lost after 35 d (FIGS. 4A and 4B). This was also observed by quantifying mass loss during this time: the PLLA scaffold lost only about 15% of its initial mass, the PLLA/PSLA with no heparin and the PLLA scaffold with heparin lost about 20-25%, and the PLLA/PSLA scaffold with conjugated heparin lost about 52% of the total mass. The nanoarchitecture observed at day 0 and at day 35 showed that the nanofibers and overall morphology had changed considerably in the heparin-modified PLLA/PSLA blend but did not change significantly in the other groups (FIG. 4C).

Thus, Example 5 demonstrates the advantageous morphology and biodegradation of the copolymers of the disclosure and scaffolds prepared therefrom relative to known polymers and scaffolds.

### Example 6—In Situ Implantation of Tubular Scaffold as a Replacement for Descending Aorta in Rat

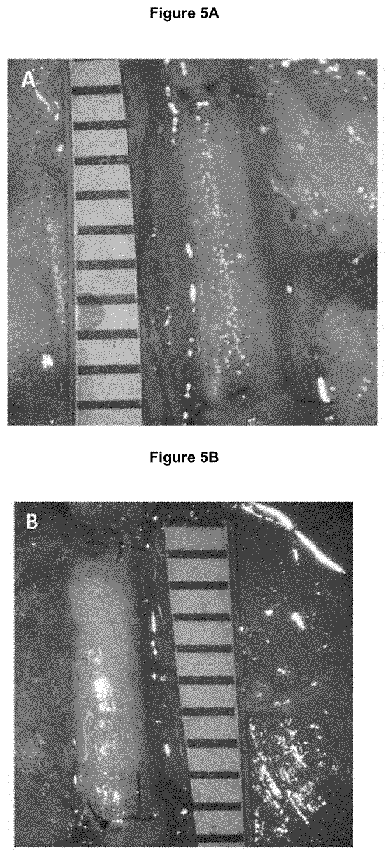

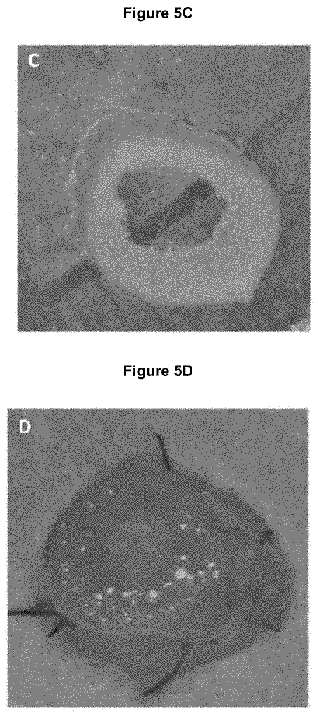

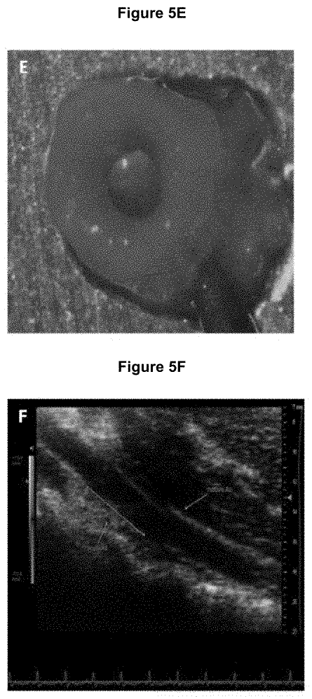

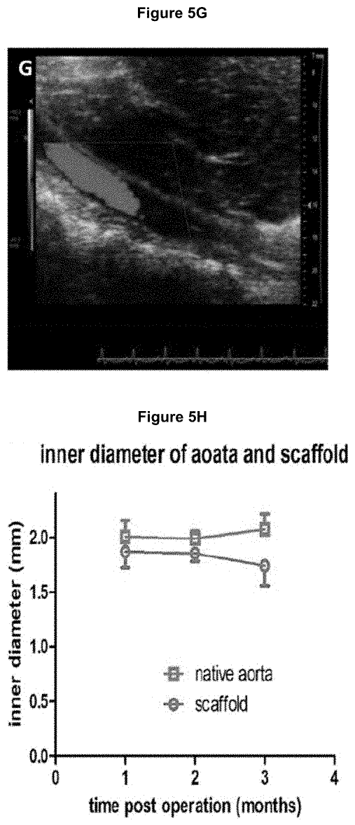

Biodegradable copolymer scaffolds 10 mm in length and 1 mm in inner diameter were prepared as described in Example 3 and functionalized as described in Example 4. The biodegradable copolymer scaffolds were in situ implanted into Sprague Dawley (SD) rats, 2-4 month-old, weighing 200-400 g (Charles River Laboratories, Boston, Mass.) as abdominal aorta interposition grafts to evaluate the viability and effectiveness of the tubular scaffolds and to determine the remodeling in vivo (FIG. 5A). All procedures were approved by the Institutional Animal Care and Use Committee at The University of Michigan. After anesthesia with ketamine (80 mg/kg) and xylazine (8 mg/kg), heparin was administered 150 units/kg intravenously through the tail vein. The animal was placed in the supine position on a warming pad (37° C.) and a midline laparotomy was performed. The infrarenal aorta was dissected and clamped using two microclamps. The vascular scaffold was implanted in an end-to-end interrupted anastomotic pattern using 9-0 prolene sutures under a microscope. The abdomen was then closed in multiple layers. Lovenox was given 100 units/kg twice a day subcutaneously for anticoagulation. The rats were sacrificed at predetermined times of 1 week, 2 weeks, 1 month, and 3 months. All rats survived to time of sacrifice with no signs of bleeding, rupture, or mechanical failure of the vessel (FIG. 5B). Importantly, there was no discoloration of the vessel, darkening of surrounding tissue, and fascia had grown around the regenerated vessels signifying an absence of either necrosis or trigger of an immune response. Additionally, the tissue excised at three months looked similar to that of the natural vasculature (FIG. 5C-FIG. 5E). After implantation, the scaffolds were observed for blood flow obstruction internal diameter changes, wall size changes, and any arising thrombosis issues via laser Doppler ultrasound and ultrasound imaging (FIG. 5F and FIG. 5G). Through these means, it was determined that blood flowed unobstructed, the wall size and internal diameter remained nearly identical. Additionally, the loose layer (i.e., the inner layer of the bifunctional nanofibrous scaffold, such as in FIG. 2B began to degrade at 2 weeks and remodeling began within 1 month as observed by a slight increase in diameter of the in situ implanted graft (FIG. 5H). Likewise, it was determined that the in situ implanted scaffolds did not form aneurysms or hyperplasia. The inner portion of the nanofibrous scaffold, such as shown in the middle images of FIG. 2B, is termed herein the “loose layer” which refers to its less dense, porous and interconnected nature. The outer portion of the nanofibrous scaffold, such as shown in the first image, far left in FIG. 2C, is termed the “dense layer” which refers to its high density of polymers. When referring to the bilayer structure of the nanofibrous scaffold disclosed herein, the terms “inner layer” and “loose layer” are used interchangeably herein. When referring to the bilayer structure of the nanofibrous scaffold disclosed herein, the terms “outer layer” and “dense layer” are used interchangeably herein

### Doppler and Ultrasound Visualization and Analysis of In Situ Graft

At a predetermined time of 1, 2, and 3 months after implantation, animals were examined using a Vevo 770® Micro-ultrasound System (Visual-Sonics, Toronto, Canada) (or equivalent) equipped with the RMV-704 scanhead (spatial resolution 40 mm) (or equivalent) to determine graft patency and blood flow. The diameter of the graft at the midpoint was measured from both transverse and longitudinal axis ultrasound images. Grafts were explanted at 1 week, 2 weeks, 1 month, 2 month, and 3 months post-operatively.

### Histological Observation of Remodeled Tissue Engineered Blood Vessels (TEBVs)

The TEBVs were washed with PBS prior to being fixed with 3.7% formaldehyde in PBS and left overnight to react. Subsequently they were dehydrated through the use of ethanol, embedded in paraffin, and sectioned at a thickness of 5 mm. Sections were deparaffinized, rehydrated with a graded series of ethanol, and stained with H-E, Masson's trichrome, and Verhoeff-Van Gieson method.

### Characterization and Assessment of Explanted Tissue Engineered Vessels

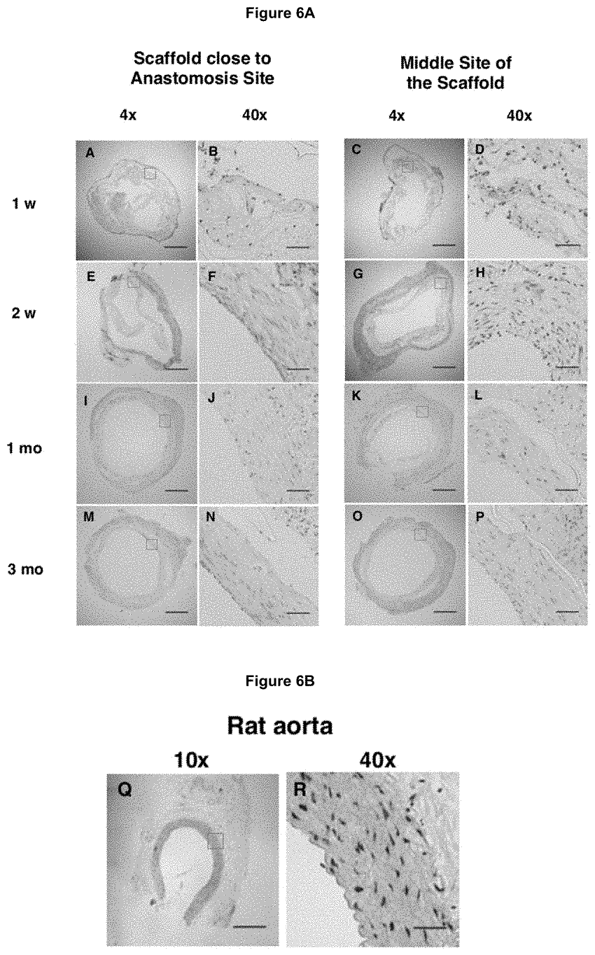

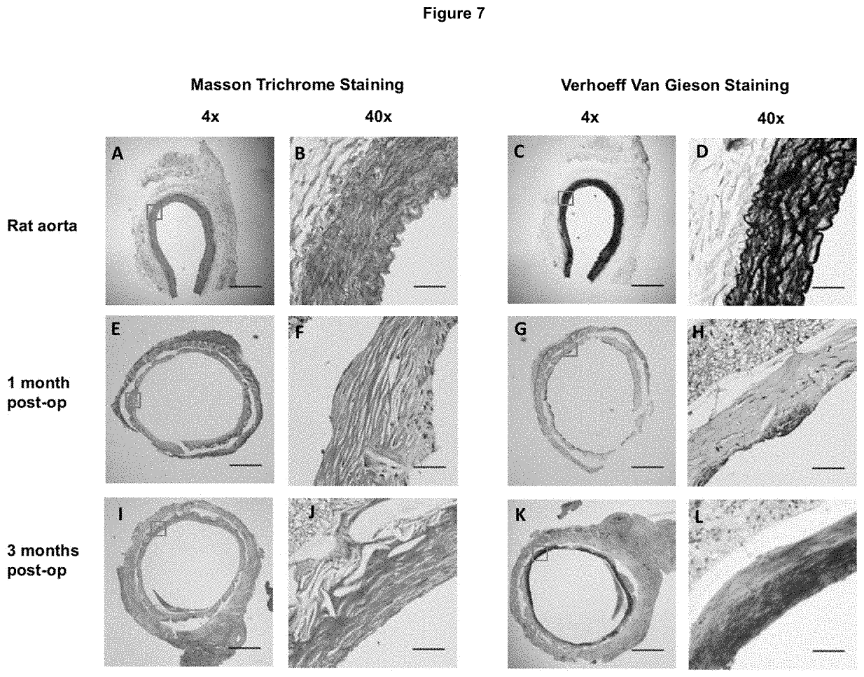

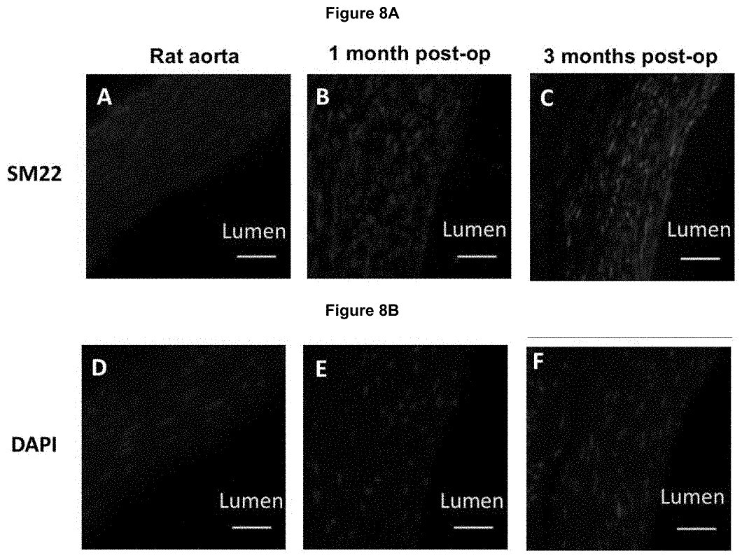

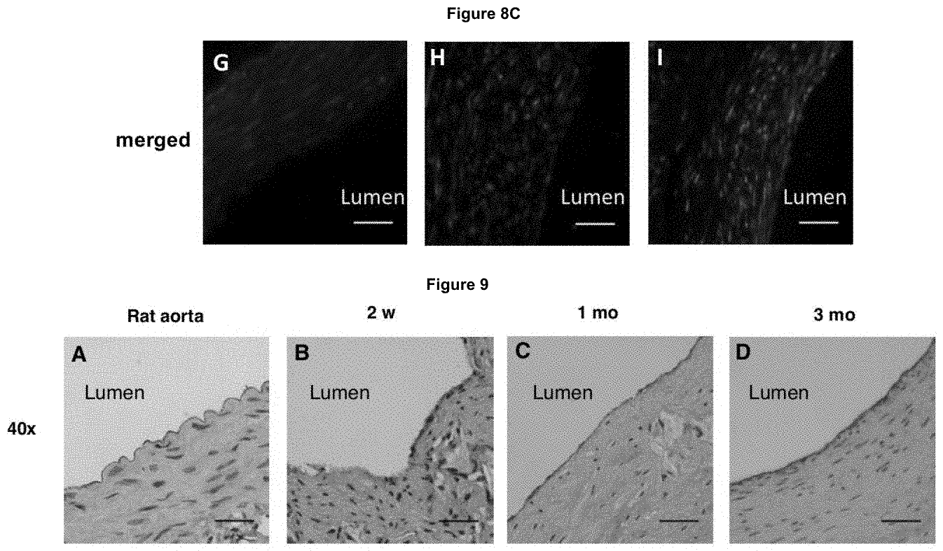

The porous and interconnected scaffolds were infiltrated by native cells after as little as one week, resulting in the degradation of the polymer and the remodeling of the inner layer. After one month of in situ implantation, the interior of the scaffold had mostly degraded, been replaced with native cells forming complex tissue configuration (FIGS. 6A and 6B). Hematoxylin and eosin staining (H&E) of the vascular graft at 1 week, 2 weeks, 1 month, and 3 months demonstrated the effectiveness of the scaffold design to promote cellular infiltration, migration and proliferation. Through these images, proliferating and migrating cells were observed within the scaffold and tissue was formed with cells orientated in a laminar fashion, similar to natural vasculature. The deposition of collagen and elastin in the newly formed tissue at 1 and 3 months was illustrated using Masson's Trichrome and Verhoeff-Van Gieson staining methods (FIG. 7). The staining demonstrated that a high quantity of collagen was deposited after one month and a high quantity of elastin was deposited after three months, with both ECM proteins approaching native tissue levels. Immunofluorescence staining was utilized to illustrate SM22, a marker protein secreted by smooth muscle cells, showing cells occupying the remodeled scaffold to be smooth muscle cells. This indicated that smooth muscle cells have infiltrated the scaffold and regenerated the rat aorta at 1 month and 3 month post-op (FIG. 8A-8C). Endothelization of the vascular graft was important for long-term viability and for the formation of mature tissue. Immunohistochemistry was done using von Willebrand factor (vWf) to demonstrate the effectiveness of the scaffold's ability for endothelization, with incomplete endothelization at 2 weeks and complete endothelization at one and three months (FIG. 9). The nanofibrous scaffold synthesized herein is the first tissue engineered graft that is able to be infiltrated by native cells, after as little as a week; to have the interior of the scaffold such that the concentration of monomers in the solution was maintained at 5% w/v one and three months.

Thus, Example 6 demonstrates successful implantation of a nanofibrous scaffold of the disclosure including a biodegradable copolymer of the disclosure and the successful regeneration of tissue according to methods of the disclosure.

1. A biodegradable copolymer comprising a structure of: 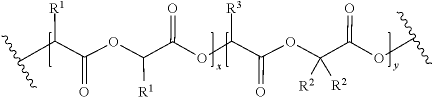
wherein
each R¹ is independently selected from C₁-C₂₂ alkyl, C₂-C₂₂ alkenyl, C₅-C₁₂cycloalkyl, C₅-C₁₂cycloalkenyl, Ar¹, carboxyl, ester, amino, thiol, halo, C₁-C₂₂ haloalkyl, SH, OH, or a first functional group of a click chemistry reactive pair;
the geminal R² groups as a pair, together with the carbon atom to which they are attached, form a five- to twelve-member cyclic or bicyclic group, or each R² is independently selected from H, C₁-C₂₂ alkenyl, C₅-C₈cycloalkenyl, carboxyl, amido, C₁-C₂₂ haloalkenyl, OH, SH, and Ar¹, or a first functional group of a click chemistry reactive pair;
R³ is selected from C₁-C₂₂ alkyl, C₂-C₂₂ alkenyl, C₅-C₁₂cycloalkyl, C₅-C₁₂cycloalkenyl, Ar¹, carboxyl, ester, amino, thiol, halo, C₁-C₂₂ haloalkyl, OH, or a first functional group of a click chemistry reactive pair;
each Ar¹ is independently selected from C₆-C₂₂ aryl or a 5-12 membered heteroaryl comprising from 1 to 3 ring heteroatoms selected from O, N, and S; and
each of x and y is an integer;
with a proviso that when the geminal R² groups as a pair, together form 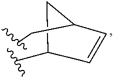 [SMILES: *CC1C=CCC(*)C1]
each occurrence of R¹ are C₁-alkyl, and R³ is C₁-alkyl, then the weight average molecular weight of the biodegradable copolymer is about 35 kDa or more.

2.-23.

24. A method of lactone polymerization comprising:
admixing:
(a) a cooled mixture comprising lactone monomers and a solvent, the mixture having a temperature of about −30° C. to about −110° C.; and
(b) about 0.01 mol % to about 5 mol % of a guanidine derivative, based on the total mols of monomers, optionally in a solvent, to form the lactone polymer, wherein upon admixing, the concentration of lactone monomers is about 15 w/v % or less, based on the total volume of solvent.

25. (canceled)

26. The method of claim 24, wherein the lactone monomers comprises a first lactone or derivative thereof, and the first lactone or derivative thereof is selected from the group consisting of: 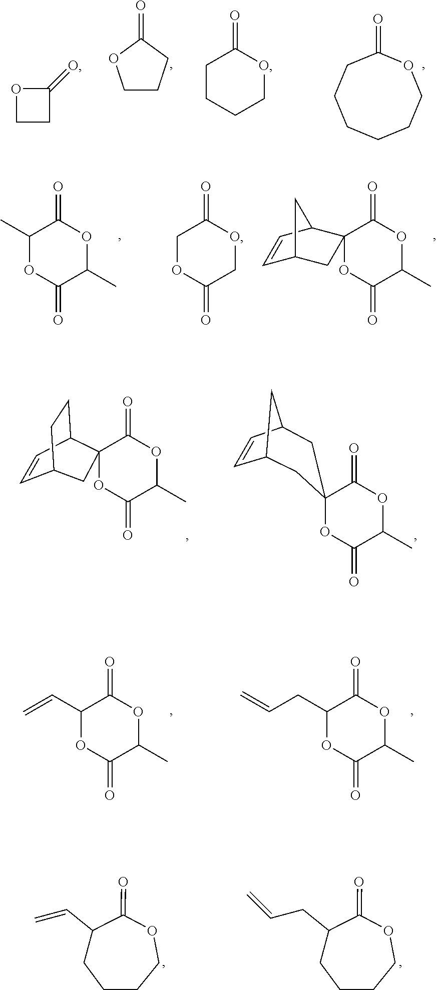
and combinations thereof.

27. The method of claim 25, wherein the mixture of monomers further comprises a second lactone or a derivative thereof.

28. The method of claim 27, wherein the first lactone is selected from the group consisting of 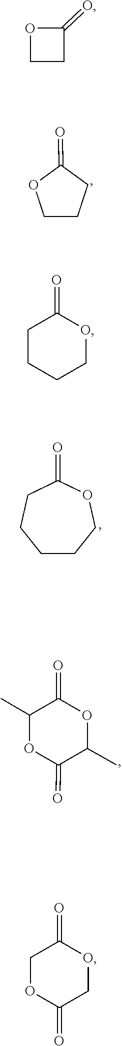
and combinations thereof.

29. The method of claim 27, wherein the second lactone is selected from the group consisting of 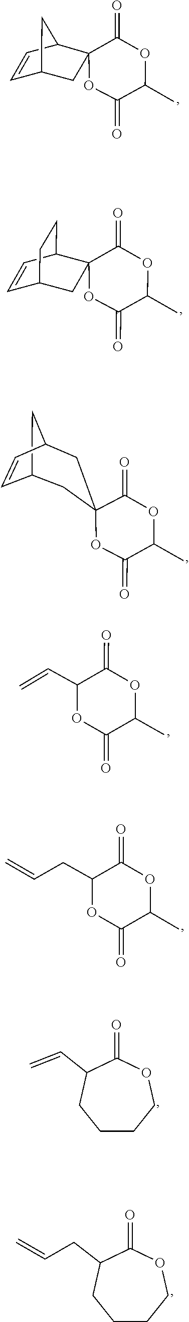
and combinations thereof.

30. The method of claim 27, wherein the first lactone comprises 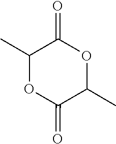
and the second lactone comprises

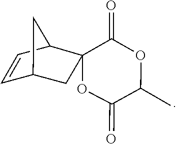

31. (canceled)

32. The method of claim 27, wherein the first lactone or derivative thereof and a second lactone or derivative thereof are provided in a ratio of about 1:1 to about 100:1.

33. (canceled)

34. The method of claim 27, further comprising: (c) admixing a second mixture of monomers to the mixture of (b).

35. The method of claim 34, wherein the second mixture of monomers comprises the first lactone or derivative thereof, the second lactone or a derivative thereof, a third lactone or a derivative thereof, or a combination thereof.

36. The method of claim 24, wherein step (a) the mixture is cooled to a temperature of about −70° C. to about −90° C.

37. The method of claim 24, wherein step (a) the mixture is cooled for about 30 minutes.

38. The method of claim 24, wherein the guanidine derivative is represented by a structure of Formula I: 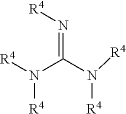
or a salt thereof, wherein each R⁴, independently, is H, C₁-C₁₀ alkyl, C₅-C₈cycloalkyl, carboxyl, ester, amino, thiol, halo, C₁-C₂₂ haloalkyl, OH, and Ar² or two R⁴ groups, together with the atoms to which they are attached, form a five- to eight-member cyclic group, wherein each Ar² is independently selected from C₆-C₂₂ aryl or a 5-12 membered heteroaryl comprising from 1 to 3 ring heteroatoms selected from O, N, and S.

39. The method of claim 24, wherein the guanidine derivative is selected from the group consisting of 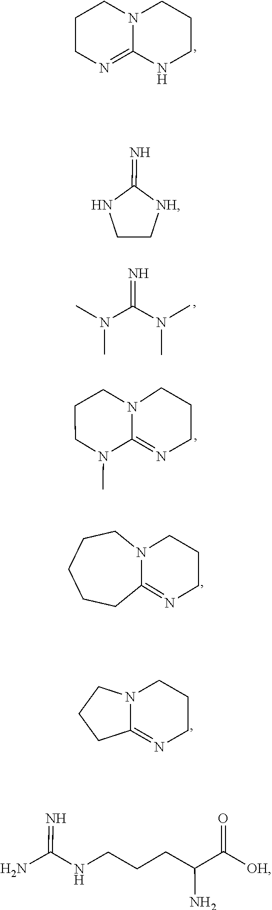
a combination thereof, and salts thereof.

40. The method of claim 24, wherein the guanidine derivative is present in an amount of about 0.05 mol % to about 0.2 mol %.

41. The method of claim 24, wherein the mixture of step (b) has a concentration of about 8 w/v % to about 0.1 w/v %.

42. The method of claim 24, wherein step (b) further comprises an initiator comprising propanol amine, hydroxyethylmethacrylate, 2-hydroxyethyl acrylate, benzyl alcohol, propargyl alcohol, propanol bromide, 2,2-bis(bromomethyl)-1,3-propanediol, pentaerythritol, pentaerythritol triacrylate, a first functional group of a click chemistry reactive pair, or a combination thereof.

43. The method of claim 24, wherein the cooled mixture and the guanidine derivative are admixed for about 1 hour to about 24 hours.

44. The method of claim 24, wherein the biodegradable polymer has a molecular weight of about 35 kDa to about 1000 kDa.

45. (canceled)

46. (canceled)

47. The method of claim 25, wherein the mixture of monomers, the guanidine derivative, or both further comprise a solvent.

48. The method of claim 47, wherein the guanidine derivative is at least partially soluble in the solvent at the temperature of the cooled mixture.

49. The method of claim 47, wherein the biodegradable polymer is substantially soluble in the solvent at the temperature of the cooled mixture.

50. The method of claim 47, wherein the solvent comprises an aprotic organic solvent.

51. (canceled)

52. A nanofibrous scaffold comprising the biodegradable copolymer of claim 1, polyspirolactide, or a combination thereof.

53.-71. (canceled)

72. A method of regenerating tissue comprising implanting the nanofibrous scaffold of claim 52 in a tissue or connected to a tissue.

73.-75. (canceled)
